SEQUENCE LISTING (35 sequences)

SEQ ID NO: 1 - protein, 428 aa, Homo sapiens
ASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 2 - protein, 46 aa, Artificial Sequence
  JUN polypeptide
CGGRIARLEEKVKTLKAQNSELASTANMLREQVAQLKQKVMNHVGC

SEQ ID NO: 3 - protein, 46 aa, Artificial Sequence
  FOS polypeptide
CGGLTDTLQAETDQVEDEKSALQTEIANLLKEKEKLEFILAAHGGC

SEQ ID NO: 4 - protein, 183 aa, Hepatitis B virus
MDIDPYKEFGATVELLSFLPSDFFPSVRDLLDTASALYREALESPEHCSPHHTALRQAILCWGELMTLATWVGGNLEDPISRDLVVSYVNTNMGLKFRQLLWFHISCLTFGRETVIEYLVSFGVWIRTPPAYRPPNAPILSTLPETCVVRRRGRSPRRRTPSPRRRRSQSPRRRRSQSRGSQC

SEQ ID NO: 5 - protein, 185 aa, Hepatitis B virus
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 6 - protein, 152 aa, Hepatitis B virus
MDIDPYKEFGATVELLSFLPSDFFPSVRDLLDTASALYREAIESPEHCSPHHTALRQAILCWGELMTLATWVGTNLEDGGKGGSRDLVVSYVNTNMGLKIRQLLWFHISCLTFGRETVLEYLVSFGVWIRTPPAYRPPNAPILSTLPETTVV

SEQ ID NO: 7 - protein, 152 aa, Hepatitis B virus
MDIDPYKEFGATVELLSFLPSDFFPSVRDLLDTASALYREALESPEHSSPHHTALRQAILCWGELMTLATWVGTNLEDGGKGGSRDLVVSYVNTNMGLKIRQLLWFHISSLTFGRETVLEYLVSFGVWIRTPPAYRPPNAPILSTLPETTVV

SEQ ID NO: 8 - protein, 182 aa, Escherichia coli
MKIKTLAIVVLSALSLSSTTALAAATTVNGGTVHFKGEVVNAACAVDAGSVDQTVQLGQVRTASLAQEGATSSAVGFNIQLNDCDTNVASKAAVAFLGTAIDAGHTNVLALQSSAAGSATNVGVQILDRTGAALTLDGATFSSETTLNNGTNTIPFQARYFATGAATPGAANADATFKVQYQ

SEQ ID NO: 9 - DNA, 15 bp, Escherichia coli
aggaggtaaaaaacg

SEQ ID NO: 10 - protein, 21 aa, Escherichia coli
MKKTAIAIAVALAGFATVAQA

SEQ ID NO: 11 - DNA, 6 bp, Artificial Sequence
  Peptidic linker
aaasgg

SEQ ID NO: 12 - DNA, 6 bp, Artificial Sequence
  Peptidic linker
ggsaaa

SEQ ID NO: 13 - DNA, 256 bp, Artificial Sequence
  pAV1 vector
gaattcaggaggtaaaaaacgatgaaaaagacagctatcgcgattgcagtggcactggctggtttcgctaccgtagcgcaggcctgggtgggggcggccgcttctggtggttgcggtggtctgaccgacaccctgcaggcggaaaccgaccaggtggaagacgaaaaatccgcgctgcaaaccgaaatcgcgaacctgctgaaagaaaaagaaaagctggagttcatcctggcggcacacggtggttgctaagctt

SEQ ID NO: 14 - protein, 74 aa, Artificial Sequence
  pAV1 vector
MKKTAIAIAVALAGFATVAQAAAASGGCGGLTDTLQAETDQVEDEKSALQTEIANLLKEKEKLEFILAAHGGCA

SEQ ID NO: 15 - DNA, 261 bp, Artificial Sequence
  pAV2 vector
gaattcaggaggtaaaaaacgatgaaaaagacagctatcgcgattgcagtggcactggctggtttcgctaccgtagcgcaggcctgcggtggtctgaccgacaccctgcaggcggaaaccgaccaggtggaagacgaaaaatccgcgctgcaaaccgaaatcgcgaacctgctgaaagaaaaagaaaagctggagttcatcctggcggcacacggtggttgcggtggttctgcggccgctgggtgtggggatatcaagctt

SEQ ID NO: 16 - protein, 73 aa, Artificial Sequence
  pAV2 vector
MKKTAIAIAVALAGFATVAQACGGLTDTLQAETDQVEDEKSALQTEIANLLKEKEKLEFILAAHGGCGGSAAA

SEQ ID NO: 17 - DNA, 196 bp, Artificial Sequence
  pAV3 vector
gaattcaggaggtaaaaagatatcgggtgtggggcggccgcttctggtggttgcggtggtctgaccgacaccctgcaggcggaaaccgaccaggtggaagacgaaaaatccgcgctgcaaaccgaaatcgcgaacctgctgaaagaaaaagaaaagctggagttcatcctggcggcacacggtggttgctaagctt

SEQ ID NO: 18 - DNA, 204 bp, Artificial Sequence
  pAV4 vector
gaattcaggaggtaaaaaacgatggcttgcggtggtctgaccgacaccctgcaggcggaaaccgaccaggtggaagacgaaaaatccgcgctgcaaaccgaaatcgcgaacctgctgaaagaaaaagaaaagctggagttcatcctggcggcacacggtggttgcggtggttctgcggccgctgggtgtggggatatcaagctt

SEQ ID NO: 19 - protein, 4 aa, Artificial Sequence
  pAV4 vector
EFRR

SEQ ID NO: 20 - protein, 56 aa, Artificial Sequence
  pAV4 vector
KTMACGGLTDTLQAETDQVEDEKSALQTEIANLLKEKEKLEFILAAHGGCGGSAAA

SEQ ID NO: 21 - protein, 26 aa, Homo sapiens
MATGSRTSLLLAFGLLCLPWLQEGSA

SEQ ID NO: 22 - DNA, 262 bp, Artificial Sequence
  pAV5 vector
gaattcaggcctatggctacaggctcccggacgtccctgctcctggcttttggcctgctctgcctgccctggcttcaagagggcagcgctgggtgtggggcggccgcttctggtggttgcggtggtctgaccgacaccctgcaggcggaaaccgaccaggtggaagacgaaaaatccgcgctgcaaaccgaaatcgcgaacctgctgaaagaaaaagaaaagctggagttcatcctggcggcacacggtggttgctaagctt

SEQ ID NO: 23 - DNA, 261 bp, Artificial Sequence
  pAV6 vector
gaattcatggctacaggctcccggacgtccctgctcctggcttttggcctgctctgcctgccctggcttcaagagggcagcgcttgcggtggtctgaccgacaccctgcaggcggaaaccgaccaggtggaagacgaaaaatccgcgctgcaaaccgaaatcgcgaacctgctgaaagaaaaagaaaagctggagttcatcctggcggcacacggtggttgcggtggttctgcggccgctgggtgtgggaggcctaagctt

SEQ ID NO: 24 - protein, 78 aa, Artificial Sequence
  pAV6 vector
MATGSRTSLLLAFGLLCLPWLQEGSACGGLTDTLQAETDQVEDEKSALQTEIANLLKEKEKLEFILAAHGGCGGSAAA

SEQ ID NO: 25 - DNA, 44 bp, Artificial Sequence
  FOS-FOR1 oligonucleotide
cctgggtgggggcggccgcttctggtggttgcggtggtctgacc

SEQ ID NO: 26 - DNA, 44 bp, Artificial Sequence
  FOS-FOR2 oligonucleotide
ggtgggaattcaggaggtaaaaagatatcgggtgtggggcggcc

SEQ ID NO: 27 - DNA, 47 bp, Artificial Sequence
  FOS-FOR3 oligonucleotide
ggtgggaattcaggaggtaaaaaacgatggcttgcggtggtctgacc

SEQ ID NO: 28 - DNA, 18 bp, Artificial Sequence
  FOS-FOR4 oligonucleotide
gcttgcggtggtctgacc

SEQ ID NO: 29 - DNA, 27 bp, Artificial Sequence
  FOS-REV1 oligonucleotide
ccaccaagcttagcaaccaccgtgtgc

SEQ ID NO: 30 - DNA, 54 bp, Artificial Sequence
  FOS-REV2 oligonucleotide
ccaccaagcttgatatccccacacccagcggccgcagaaccaccgcaaccaccg

SEQ ID NO: 31 - DNA, 32 bp, Artificial Sequence
  FOS-REV3 oligonucleotide
ccaccaagcttaggcctcccacacccagcggc

SEQ ID NO: 32 - DNA, 29 bp, Artificial Sequence
  OmpA-FOR1 oligonucleotide
ggtgggaattcaggaggtaaaaaacgatg

SEQ ID NO: 33 - DNA, 32 bp, Artificial Sequence
  hGH-FOR1 oligonucleotide
ggtgggaattcaggcctatggctacaggctcc

SEQ ID NO: 34 - DNA, 27 bp, Artificial Sequence
  hGH-FOR2 oligonucleotide
ggtgggaattcatggctacaggctccc

SEQ ID NO: 35 - protein, 11 aa, Artificial Sequence
  Synthetic FLAG peptide with Cys residue at amino terminus
CGGDYKDDDDK

(19) United States
(12) Patent Application Publication
(10) Pub. No.: US 2002/0146422 A1
(43) Pub. Date: Oct. 10, 2002

(54) Compositions for inducing self-specific anti-IgE antibodies and uses thereof

(75) Inventors: Martin F. Bachmann, Winterhur (CH); Wolfgang A. Renner, Zurich (CH)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: Cytos Biotechnology AG

(21) Appl. No.: 09/916,230

(22) Filed: Jul. 27, 2001

Related U.S. Application Data
(60) Provisional application No. 60/221,841, filed on Jul. 28, 2000.

(51) Int. Cl. A61K 39/395

(52) U.S. Cl. 424/178.1; 424/93.6

(57) ABSTRACT

The invention relates to compositions for the induction of anti-IgE antibodies in order to prevent or inhibit IgE-mediated disorders. The compositions contain carriers foreign to the immunized human or animal coupled to polypeptides containing fragments of the IgE molecule. The fragment of the IgE molecule includes the constant CH1 and/or the CH4 domain of the IgE molecule. The composition is administered to humans or animals in order to induce antibodies specific for endogenous IgE antibodies. These induced anti-IgE antibodies reduce or eliminate the pool of free IgE in the serum. Since many allergic diseases are mediated by IgE, IgE-mediated disorders are ameliorated in treated mammals.

### CROSS-REFERENCE TO RELATED APPLICATIONS

This application claims the benefit under 35 U.S.C. §119(e) of U.S. Provisional Application No. 60/221,841, filed on Jul. 28, 2000, herein incorporated by reference in its entirety.

### BACKGROUND OF THE INVENTION

1. Field of the Invention

This invention relates to methods and compositions for inducing the production of antibodies that specifically bind to endogenous IgE. More particularly, the invention relates to methods and compositions for inhibiting or preventing IgE-mediated disorders.

2. Related Art

The number of people suffering from allergic reactions is rapidly increasing in the western world. Indeed, 10-20% of the population can be considered to suffer from an allergy. A major cause of allergic reactions is the recognition of allergens by IgE antibodies. Upon binding of IgE to receptors on mast cells and basophils, highly active substances such as histamine, leukotrines, platelet activating factor, heparin, chemotactic factors, and prostaglandins are rapidly released, causing IgE-mediated allergic reactions (Type I hypersensitivity). These reactions include various forms of asthma; allergies to pollen, fur, and/or house dust; various food allergies; and various forms of eczema.

To trigger an allergic reaction, IgE antibodies must bind to receptors on mast cells or basophils. Previous attempts to use short peptides or small molecules to inhibit the interaction of IgE with its receptor, and thus inhibit allergic reactions, have not been very successful, due to stability or toxicity problems. Monoclonal antibodies that specifically bind to CH3 domains of IgE have been administered to mammals to inhibit binding of IgE to its receptor. In human clinical trials, such monoclonal antibodies ameliorated allergic reactions. However, treatment with monoclonal antibodies requires the long-term, and possibly life-long, administration of the monoclonal antibodies. In addition, treatment with monoclonal antibodies may produce side effects, such as the induction of antibodies that specifically bind to the therapeutic monoclonal antibodies.

Detailed studies of the interaction of the IgE molecule with the high-affinity receptor for IgE have shown that a region of 76 amino acids at the border between the CH2 and CH3 domains (i.e., constant domains 2 and 3 in the heavy chain) of IgE is important for the interaction between the IgE molecule and its high-affinity receptor. This peptide has been shown, in vitro, to be able to inhibit the interaction between native IgE and its high-affinity receptor.

### SUMMARY OF THE INVENTION

The invention is derived, at least in part, from the discovery that a polypeptide that includes a CH1 and/or CH4 domain(s) of an IgE molecule, coupled to a carrier, can be used to induce in a mammal the production of antibodies that specifically bind to IgE of the mammal. Such a composition can be used therapeutically to inhibit or treat an IgE-mediated disorder, such as an allergic reaction, in a mammal.

Accordingly, the invention features a composition comprising (i) a carrier (e.g., a polypeptide) comprising a first attachment site; and (ii) a polypeptide selected from the group consisting of (a) at least one CH1 domain of an IgE molecule; (b) at least one CH4 domain of an IgE molecule; and (c) a combination of (a) and (b); wherein the polypeptide having the IgE domain contains or is bound to a second attachment site; wherein the first and second attachment sites are bound to each other. The IgE domains optionally comprise one or more linkers covalently linking the domains. The first attachment site can be bound either directly or indirectly to the second attachment site. In one embodiment of the invention, the first attachment site is bound to a crosslinking agent which in turn is bound to the second attachment site.

Preferably, the polypeptide lacks an IgE CH3 domain. The carrier can be a virus, a virus-like particle, a bacteriophage, a bacterial pilus, a viral capsid particle, or a recombinant protein thereof. For example, the carrier can be a virus-like particle derived from, e.g., a Papilloma virus, a Rotavirus, a Norwalk virus, an Alphavirus, a Foot and Mouth Disease virus, a Retrovirus, or a Hepatitis B virus.

In one embodiment, the first and second attachment sites comprise: (a) an antigen and an antibody or antibody fragment that specifically binds thereto, (b) biotin and avidin (c) streptavidin and biotin, (d) a receptor and a ligand that binds to the receptor, (e) a ligand-binding protein and a ligand, (f) interacting leucine zipper polypeptides, (g) an amino group and a chemical group reactive therewith, (h) a carboxyl group and a chemical group reactive therewith, or (i) a sulfhydryl group or a chemical group reactive therewith. In a preferred embodiment, the first attachment site is bound to the second attachment site via a crosslinking agent. In another preferred embodiment, the crosslinking agent is a heterobifunctional crosslinking agent. In another preferred embodiment, an amino group is covalently bound to a heterobifunctional cross-linking agent which is in turn covalently bound to a sulfhydryl group.

If desired, first and second attachment sites are bound to each other via a chemically-reactive amino acid which can be part of the first or second attachment sites. Alternatively, the first attachment site is bound to the second attachment site via a peptide bond, thereby providing a fusion protein comprising the polypeptide and the carrier. In other embodiments, the first and second attachment sites comprise all or a portion of protein A; all or a portion of an immunoglobulin (Ig) variable region (preferably anon-human Ig variable region); all or a portion of protein L; or all or a portion of a rodent IgG CH2 domain and all or a portion of a rodent IgG CH3 domain. Such attachment sites can be designed to facilitate binding between (i) protein A (or a portion thereof) and IgG CH2-CH3 (or a portion thereof), or (ii) Ig variable region and protein L (or a portion thereof).

In various embodiments, the IgE-containing polypeptide comprises at least two CH4 domains and/or at least two CH1 domains, or at least two domains selected from the group consisting of a CH1 domain and a CH4 domain. The IgE-containing polypeptide further comprises one or more linkers covalently linking the domains. If desired, the polypeptide can include a CH1 domain and a CH4 domain. Preferably, the IgE molecule from which the domains are derived is a human IgE molecule. Optionally, the carrier comprises one or more epitopes of a T helper cell. Optionally, the carrier is a non-human protein. If desired, the composition can also include an adjuvant.

Various nucleic acids and cells are encompassed by the invention. For example, the invention includes a polynucleotide encoding a fusion protein that includes the IgE-containing polypeptide and the carrier fused together. The invention also includes a gene comprising this polynucleotide; a vector comprising the gene; and a cell comprising the vector or polynucleotide. The invention also includes a method for producing the fusion protein by inserting a vector containing a polynucleotide sequence encoding the fusion protein into a cell, and maintaining the cell under conditions such that the fusion protein is expressed. Also within the invention is a cell in vitro or a non-human cell that includes the composition of the invention.

The compositions and nucleic acids of the invention can be used in therapeutic methods for inhibiting or preventing IgE-mediated disorders. For example, the invention includes a method for eliciting an immune response in a mammal by administering to the mammal an immunogenic amount of the composition of the invention, or by administering to a mammal an immunogenic amount of a polynucleotide encoding a fusion protein of the invention. The invention also features a method for treating or inhibiting an IgE-mediated disorder in a mammal by administering to a mammal in need thereof an effective amount of a composition of the invention, or by administering an effective amount of a polynucleotide encoding a fusion protein of the invention.

The compositions and polynucleotides of the invention can be used to inhibit or prevent IgE-mediated disorders such as anaphylactic shock, allergic rhinitis or conjunctivitis, an allergic reaction to an allergen such as fur, dust, or food, an asthmatic reaction, eczema or urticaria.

In another aspect, the invention relates to a composition comprising (i) a carrier comprising a first attachment site; and (ii) a polypeptide selected from the group consisting of: (a) at least one CH1 domain of an IgE molecule; (b) at least one CH4 domain of an IgE molecule; and (c) a combination of (a) and (b); wherein the polypeptide having the IgE domain comprises a second attachment site; wherein the first attachment site is bound to the second attachment site; wherein the attachment sites are bound to each other via a heterobifunctional cross-linking agent; and wherein the agent comprises a N-hydroxy-succinimide ester group and a maleimide group.

The heterobifunctional cross-linking agent can be ε-maleimidocaproic acid N-hydroxy-succinimide ester. Other hetero-bifunctional cross-linkers can be used in the present invention such as, by way of example, SMCC (Succinimidyl 4-[N-maleimidomethyl]-cyclohexane-1-carboxylate), SMPB (Succinimidyl 4-p-maleimidophenyl]-butyrate), (N-[γ-Maleimidobutylody]sulfosuccinimide ester), Sulfo-SMCC (Sulfosuccinimidyl 4 [N-maleimidomethyl]-cyclohexane- 1-carboxylate), Succinimidyl-3-[bromoacetamido] propionate and SIAB (from the supplier Pierce) can also be used in making compositions of the invention.

An amino moiety in the first attachment site reacts with the N-hydroxy-succinimide ester group; and the maleimide group is chemically coupled to the thiol moiety of a cysteine group on the second attachment site.

Alternatively, an amino moiety of the second attachment site reacts with the N-hydroxy-succinimide ester group; and the maleimide group is chemically coupled to the thiol moiety of a cysteine group on the attachment site.

In another aspect, the invention relates to a cell comprising at least one isolated polypeptide selected from the group consisting of: (a) one or a plurality of CH1 domains of an IgE molecule; (b) one or a plurality of CH4 domains of an IgE molecule; and (c) a combination of one or a plurality of CH1 domains of an IgE molecule and one or a plurality of CH4 domains of an IgE molecule. As used herein, an isolated polypeptide is one that is not contiguous with either the N-terminal or C-terminal (upstream or downstream) sequences with which the polypeptide is naturally contiguous. In a preferred embodiment of this cell, the polypeptide consists of one or a plurality of CH1 domains of an IgE molecule, wherein each of the one or a plurality of CH1 domains is an amino acid sequence at least 95% identical to a sequence selected from the group consisting of: (a) amino acids 1-110 of SEQ ID NO:1; (b) amino acids 1-105 of SEQ ID NO:1; (c) amino acids 5-105 of SEQ ID NO:1; and (d) amino acids 5-95 of SEQ ID NO:1. In another preferred embodiment of the cell, the polypeptide consists of one or a plurality of CH4 domains of an IgE molecule, wherein each of the one or a plurality of CH4 domains is an amino acid sequence at least 95% identical to a sequence selected from the group consisting of: (a) amino acids 313-428 of SEQ ID NO:1; (b) amino acids 313-425 of SEQ ID NO:1; (c) amino acids 317-428 of SEQ ID NO:1; and (d) amino acids 317-425 of SEQ ID NO:1. In another preferred embodiment of this cell, the polypeptide consists of the combination, wherein the combination consists of

(i) one or a plurality of CH1 domains of an IgE molecule, wherein each of the one or a plurality of CH1 domains is an amino acid sequence at least 95% identical to a sequence selected from the group consisting of:

(a) amino acids 1-110 of SEQ ID NO:1;

(b) amino acids 1-105 of SEQ ID NO:1;

(c) amino acids 5-105 of SEQ ID NO:1; and

(d) amino acids 5-95 of SEQ ID NO:1;

and

(ii) one or a plurality of CH4 domains of an IgE molecule, wherein each of the one or a plurality of CH4 domains is an amino acid sequence at least 95% identical to a sequence selected from the group consisting of:

(a) amino acids 313-428 of SEQ ID NO:1;

(b) amino acids 313-425 of SEQ ID NO:1;

(c) amino acids 317-428 of SEQ ID NO:1; and

(d) amino acids 317-425 of SEQ ID NO:1.

Alternatively, in another preferred embodiment of the cell, the CH1 and CH4 domains are about 96%, 97%, 98%, 99% and 100% identical to the above sequences, respectively.

The invention offers several advantages. The compositions of the invention are expected to induce anti-IgE responses in the presence of high levels of endogenous IgE. An alternative composition would additionally induce cytotoxic T cells recognizing IgE-derived polypeptides. The compositions of the invention also can be expected to induce the production of antibodies that specifically bind to IgE without inducing an allergic reaction against the composition itself. In addition, polyclonal B cell responses against whole domains of IgE are expected to be more efficient than B cell responses against single peptide epitopes on IgE, since this would facilitate clearance of IgE from the body. Compositions of the invention that include viral-based carriers induce prompt and efficient immune responses in the absence of any adjuvants both with and without T-cell help (Bachmann & Zinkemagel, Ann. Rev. Immunol 15:23 5-270 (1997)). Although viruses often consist of few proteins, they are able to trigger much stronger immune responses than their isolated components. For B-cell responses, it is known that one significant factor affecting the immunogenicity of viruses is the repetitiveness and order of surface epitopes. Many viruses exhibit a quasi-crystalline surface that displays a regular array of epitopes which efficiently crosslinks epitope-specific immunoglobulins on B cells (Bachmann & Zinkernagel, Immunol. Today 17:553-559 (1996)). This crosslinking of surface immunoglobulins on B cells is a strong activation signal that directly induces cell-cycle progression and the production of IgM antibodies. Further, such triggered B cells are able to activate T helper cells, which in turn induce a switch from IgM to IgG antibody production in B cells and the generation of long-lived B cell memory —the goal of any vaccination (Bachmann & Zinkernagel, Ann. Rev. Immunol. 15:235-270 (1997)). Viral structure is even linked to the generation of antibodies in autoimmune disease and as a part of the natural response to pathogens (see Fehr, T., et al, J. Exp. Med. 185:1785-1792 (1997)). Thus, antibodies presented by a highly organized viral carrier are able to induce strong anti-antibody responses. In addition to strong B cell responses, viral particles are also able to induce the generation of a cytotoxic T cell response, another important arm of the immune system. Cytotoxic T cells recognizing IgE-derived polypeptides may eliminate IgE producing B cells, further reducing levels of endogenous IgE.

Tolerance of the immune system against self-derived structures may be broken by coupling the self-antigen (i.e., an IgE-containing polypeptide) to a carrier that can deliver T help. For soluble proteins present at high concentrations or membrane proteins at low concentration, B and Th cells may be tolerant. However, B cell tolerance can be broken by administration of the IgE-containing polypeptide in a highly organized fashion coupled to a foreign carrier, as described herein.

### DETAILED DESCRIPTION OF THE INVENTION

The invention provides compositions that can be used to inhibit or treat IgE-mediated disorders in a mammal. The compositions of the invention include a carrier having a first attachment site and a polypeptide that includes at least one of (i) a CH1 constant domain of an IgE molecule and (ii) a CH4 constant domain of an IgE molecule. The IgE-containing polypeptide also includes a second attachment site to facilitate coupling of the polypeptide to a first attachment site present in a carrier. The IgE-containing polypeptide contains or is bound to the second attachment site. As used herein. “bound” refers to covalent bonds or non-covalent interatomic or intermolecular interactions. As used herein, “first attachment site” refers to an attachment site on the carrier; and “second attachment site” refers to an attachment site on the IgE-containing polypeptide. In polypeptides that include multiple IgE domain(s), the domains optionally are linked to each other by linkers. The composition of the invention also includes a carrier (e.g., a polypeptide, virus, pilin, or virus-like particle) that includes a first attachment site. The second attachment site on the IgE-containing polypeptide is bound to the first attachment site on the carrier. The first attachment site can be bound either directly or indirectly to the second attachment site. In one embodiment of the invention, the first attachment site is bound to a crosslinking agent which in turn is bound to the second attachment site.

The entire CH1 and/or CH4 domain is included in the polypeptide. Such a polypeptide is referred to herein as an IgE-containing polypeptide. The CH1 domain relevant to the invention should preferably comprise amino acids 1-110 or 1-105 or 5-105, or 5-95 of the sequence of the human IgE epsilon chain C region (SEQ ID NO:1: ASTQSPSVFPLTRCCKNIPSNATSVTLGCLATGY FPEPVMVTWDTGSLNGTTMTLPATTLTLSGHYATISLLTVSGAWAK QMFTCRVAHTPSSTDWVDNKTFSVCSRDFTPPTVKILQSSCDGGGHFPPT IQLLCLVSGYTPGTINITWLEDGQVMDVDLSTASTTQEGELASTQSELTL SQKHWLSDRTYTCQVTYQGHTFEDSTKKCADSNPRGVSAY LSRPSPFDLFIRKSPTITCLVVDLAPSKGTVNLTWSRASGKPVNHSTRKE EKQRNGTLTVTSTLPVGTRDWIEGETYQCRVTHPHLPRAL MRSTTKTSGPRAAPEVYAFATPEWPGSRDKRTLACLIQNFMPEDISVQWL HNEVQLPDARHSTTQPRKTKGSGFFVFSRLEVTRAEWEQKDEFICRAVHE AASPSQTVQRAVSVNPGK; NCBI accession EHHU; PID g70024; PIR Database). Alternatively, the CH1 domain can be about 95%, 96%, 97%, 98% or 99% identical to amino acids 1-110 or 1-105 or 5-105, or 5-95 of the sequence of the human IgE epsilon chain C region (SEQ ID NO:1). The sequence disclosed here is representative of all human IgE sequences. There may, however, be allelic differences and some amino acids may vary between alleles. The degree of identity is, however, such that a sequence alignment with the sequence disclosed here will teach which residues to chose in the corresponding allele. In the case where the variants comprising residue 105 are chosen for preparing the composition of the invention, residue 105 fulfills the function of a second attachment site. The CH4 domain should preferably comprise residues 313-428, or 313-425, or 317-428, or 317-425 of the human IgE epsilon chain C region (See SEQ ID NO:1; NCBI accession EHHU; PID g70024; PIR Database). Alternatively, the CH4 domain can be about 95%, 96%, 97%, 98% or 99% identical to amino acids 313-428, or 313-425, or 317-428, or 317-425 of the sequence of the human IgE epsilon chain C region (SEQ ID NO:1). Typically, the polypeptide lacks a human IgE CH3 domain. The human epsilon constant region locus has been described (see, e.g., Max et al., Cell 29:691 (1982)). Thus, persons of ordinary skill in the art can readily use conventional molecular biology techniques to produce the IgE-containing polypeptides used in compositions of the invention. Various combinations of CH1 and/or CH4 domains can be used to produce the compositions of the invention. For example, two or more CH4 domains can be linked together (e.g., CH4-CH4 or CH4-CH4-CH4), a CH4 domain can be linked to a CH1 domain (e.g., CH4-CH1), or two or more CH1 domains can be linked together (e.g., CH1-CH1 or CH1-CH1-CH1-CH1). Other combinations of CH1 and/or CH4 domains can be used in the invention. In various embodiments, the polypeptide of the invention includes at least 1 (e.g., 2, 3, 4, 5, 10, 15, or even more) CH1 and/or CH4 domains linked together. Preferably, the CH1 and/or CH4 domains are derived from an IgE molecule of the same species as the mammal to be treated. For example, CH1 and/or CH4 domains of a human IgE molecule are preferred for use in methods for treating humans. In other embodiments, the IgE molecule may be derived from non-human mammals, such as, without limitation, rodents (e.g., mice or rats), non-human primates (e.g., monkeys, chimpanzees), cattle or domesticated mammals (e.g., horses, dogs, cats, guinea pigs).

In other exemplary compositions of the invention, the polypeptide includes a variable region of an immunoglobulin (Ig) light chain. For example, a CH4 domain can be linked to the variable region of a human or non-human Ig light chain (CH4-Vκ). In an alternative composition, the CH4 domain(s) is linked to the CH2-CH3 domain of IgG, preferably a rodent (e.g., mouse or rat) CH2-CH3 domain (CH4-(CH2-CH3)_{m/r}). In other exemplary compositions, a CH1 domain is fused to a variable region of a human or non-human Ig light chain (CH1-Vκ), or the CH1 domain is fused to a rodent CH2-CH3 domain of IgG (CH1-(CH2-CH3)_{m/r}). Other exemplary compositions include, without limitation, polypeptides such as the following: CH1-CH4-Vκ, CH4-CH1-Vκ, CH1-CH4-(CH2-CH3)_{m/r}, and CH4-CH1-(CH2-CH3)_{m/r}.

Nucleic acid sequences encoding the CH1 and CH4 domains have been cloned and can readily be used by persons of ordinary skill in the art of molecular biology to produce the compositions of the invention (see, e.g., Ishida et al., EMBO J. 1:1117-1123(1982) and Seno et al., Nucleic Acids Research 11:719 (1983)). In addition, nucleic acid sequences encoding the CH2-CH3 domain and the variable region of Ig light chain also have been cloned (see, e.g., Miyata et al., Proc. Nat'l. Acad. Sci. 77:2143 (1980) and Wu et al., Proc. Nat'l. Acad. Sci. 76:4617 (1979)).

Optionally, the IgE-containing polypeptide includes one or more linkers, covalently linking the immunoglobulin domains to each other. Such linkers typically are polypeptides of, e.g., 2 to 100 (e.g., 10 to 50) amino acids in length. The amino acid sequence of the linker is not critical, provided that the linker is flexible and assumes an unstructured configuration in an aqueous solution. Conventional methods can be used to produce linkers that are suitable for use in the invention. For example, the computer program LINKER can be used to design suitable linkers (Crasto and Feng, Protein Eng. 13:309-312 (2000); http://www.fccc.edu/research/labs/feng/link.html). Other examples of suitable methods for producing linkers are described in U.S. Pat. Nos. 5,990,275 and 5,856,456, which are incorporated herein by reference. Further, an amino acid spacer may be inserted between the antigen and the second attachment site.

The IgE-containing polypeptide also contains a second attachment site to facilitate binding of the polypeptide to a carrier. The second attachment site may be naturally present in the IgE-containing polypeptide, or the IgE-containing polypeptide may be engineered to contain such an attachment site. The second attachment site is an element to which a first attachment site of the carrier can bind. The second attachment site may be a protein, a polypeptide, a sugar, a polynucleotide, a natural or synthetic polymer, a metabolite or compound (e.g., biotin, fluorescein, retinol, digoxigenin, metal ions, phenylmethylsulfonyl fluoride), or a combination thereof, or a chemically reactive group thereof. For example, the second attachment site may include an antigen, an antibody or antibody fragment, biotin, avidin, streptavidin, a ligand, a ligand-binding protein, an interacting leucine zipper polypeptide, an amino group, a chemical group reactive to an amino group; a carboxyl group, a chemical group reactive to a carboxyl group, a sulfhydryl group, a chemical group reactive to a sulfhydryl group, or a combination thereof. In a preferred embodiment the second attachment site is a portion of an immunoglobulin (e.g., a rodent CH2-CH3 region or a variable region of an Ig light chain) to which a polypeptide binds (e.g., protein A or protein L).

The compositions of the invention also include a carrier, which includes a first attachment site that binds to the second attachment site of the IgE-containing polypeptide. The “carrier” comprises a polypeptide, a virus, a virus-like particle, a bacteriophage, a bacterial pilus, or a viral capsid protein, or a recombinant protein thereof. For example, the carrier can include a recombinant protein(s) of a Rotavirus, a Norwalk virus, an Alphavirus, a Foot and Mouth Disease virus, a Retrovirus, a Hepatitis B virus (e.g., a HBcAg), a Tobacco mosaic virus, a Flock House Virus, or a human Papillomavirus. Alternatively, the carrier can include a protein(s) that forms a bacterial pilus or a pilus-like structure.

In various embodiments, the carrier comprises a virus, a bacterial pilus, a structure formed from bacterial pilin, a bacteriophage, a virus-like particle, or a viral capsid particle. Any virus having a coat and/or core protein with an ordered and repetitive structure can be used as a carrier. Examples of suitable viruses include Sindbis and other Alphaviruses, vesicular stomatitis virus, rhabdovirus, picornavirus, togavirus, orthomyxovirus, polyomavirus, parvovirus, rotavirus, Norwalk virus, Foot and Mouth Disease virus, retroviruses, Hepatitis viruses, Tobacco mosaic virus, Flock House Virus, and human papillomavirus (for example, see Table 1 in Bachman, M. F. and Zinkernagel, R. M., Immunol. Today 17:553-558 (1996)).

In a preferred embodiment, the carrier is a recombinant Alphavirus, and more specifically, a recombinant Sindbis virus. Alphaviruses are positive stranded RNA viruses that replicate their genomic RNA entirely in the cytoplasm of the infected cell and without a DNA intermediate (Strauss, J. and Strauss, E., Microbiol. Rev. 58:491-562 (1994)). The alphaviral carrier of the invention may be constructed by means generally known in the art of recombinant DNA technology (See, e.g., Xiong, C. et al., Science 243:1188-1191 (1989); Schlesinger, S., Trends Biotechnol. 11:18-22 (1993); Liljeström, P. & Garoff, H., Bio/Technology 9:1356-1361 (1991); Davis, N. L. et al., Virology 171:189-204 (1989); Lundstrom, K., Curr. Opin. Biotechnol. 8:578-582 (1997); Liljeström, P., Curr. Opin. Biotechnol. 5:495-500 (1994); Boorsma et al., Nat. Biotech. 18:429 (2000) and U.S. Pat. Nos. 5,766,602; 5,792,462; 5,739,026; 5,789,245 and 5,814,482, each of which is incorporated herein by reference).

In other embodiments, the carrier is a protein of a highly organized structure, thus producing a composition in which the IgE domains are arranged in a ordered fashion. For example, the highly organized structure can be a virus or a virus-like particle (VLP). A VLP is a non-infectious, symmetrical supermolecular structure that is composed of many protein molecules of one or more types. VLPs lack a functional viral genome. Suitable VLPs can be made from proteins of viruses such as bacteriophage, Rotavirus, Norwalkvirus, Alphavirus, Foot and Mouth Disease virus, Retroviruses, Hepatitis viruses (e.g., a Hepatitis B virus), Tobacco mosaic virus, Flock House Virus, a human Papillomavirus, or a measles virus, (see, e.g., Ulrich et al., Virus Res. 50:141-182 (1998); Warnes et al., Gene 160:173-178 (1995); U.S. Pat. Nos. 5,071,651 and 5,374,426; Twomey et al., Vaccine 13:1603-1610, (1995); Jiang, X.. et al., Science 250:1580-1583 (1990); Matsui, S. M., et al., J. Clin. Invest. 87:1456-1461 (1991); PCT Patent Appl. Nos. WO 96/30523, WO 92/11291, and WO 98/15631; and Kratz, P. A., et al., Proc. Natl. Acad. Sci. USA96: 19151920 (1999)).

Other exemplary carriers that can be used in the invention includes non-toxic (preferably enzymatically inactive) polypeptides that are at least 100 amino acids in length. Examples include ovalbumin and Keyhole Limpet Hemocyanin. If desired, the carrier and the IgE-containing polypeptide can be coupled via a peptide bond formed between the first attachment site (i.e., an amino acid) in the carrier and a second attachment site (i.e., an amino acid) in the IgE-containing polypeptide. The resulting fusion protein can be used in the methods described herein for treating or inhibiting IgE-mediated disorders in a mammal.

Conventional molecular biology techniques can be used to produce the IgE-containing polypeptides and carriers used to produce the compositions of the invention. Appropriate nucleic acid sequences can be inserted into an appropriate expression vector, and the gene's native promoter may be employed or an exogenous promoter can be used. A variety of suitable promoters are available for expression in prokaryotic or eukaryotic cells. Suitable host cells include E. coli; B. subtilis; yeast cells; mammalian cells, e.g. COS cells, HeLa cells, myeloma or hybridoma cells, Sp2/0 cells, CHO cells, L(tk−−) cells, and primary cultures; insect cells; Xenopus laevis oocytes; and the like. The promoter is operably linked to the coding sequence of interest. The promoter can be either constitutive or inducible. After introduction of the nucleic acid into the host cell, the cells containing the construct may be selected by means of a selectable marker, present on the nucleic acid introduced into the cell.

The vectors that can be used in the invention may provide for extrachromosomal maintenance, particularly as plasmids or viruses, or for integration into the host chromosome. Where extrachromosomal maintenance is desired, an origin of replication can be included for the replication of the vector, e.g., a low-or high-copy plasmid. A wide variety of markers are suitable, particularly those which protect against toxins, more particularly against antibiotics. The particular marker that is chosen will be selected in accordance with the nature of the host. If desired, complementation may be employed with auxotrophic hosts, e.g., bacteria or yeast.

The DNA construct may be introduced into the cell using conventional methods, e.g. conjugation, calcium-precipitation, electroporation, fusion, transfection, infection with viral vectors, etc. Conventional cloning, expression, and genetic manipulation techniques can be used in practicing the inventions disclosed herein (see, e.g., Molecular Cloning, A Laboratory Manual (2nd Ed., Sambrook, Fritsch and Maniatis, Cold Spring Harbor) and Current Protocols in Molecular Biology (Eds. Ausubel, Brent, Kingston, Moore, Seidman, Smith and Struhl, Greene Publ. Assoc., Wiley-Interscience, NY, N.Y., 1992)).

If desired, the IgE-containing polypeptide and the carrier can be produced in bacteria, e.g., in E. coli, as a fusion protein with glutathione S-transferase as the carrier. By means of PCR (Polymerase Chain Reaction), the cDNA sequences for the CH 1 and/or CH4 regions of human IgE can be ligated into a commercially available vector for the production of a fusion protein in bacterial hosts. For example, the vector used can be one of the pGEX vectors of form 1, 2 or 3 with different reading frames for ligation of cDNA fragments (Smith and Johnson, 1988). In this vector family, the entire coding region for a 26 kD glutathione-S-transferase (Sj26) from the parasitic worm Schistosomajaponcium is cloned behind a strong and inducible tac promoter, which is negatively regulated by the lac-repressor. To obtain large amounts of protein, inhibition of the promoter is relieved by means of IPTG (isopropyl-β-D-thiogalactoside). Following ligation of the IgE coding sequence into the vector in the 3′ part of the Sj26 gene, this vector is introduced into E. coli for the production of the fusion protein. An overnight culture of the recombinant bacteria, containing the vector into which the desired sequence has been ligated, is diluted in a bacterial growth medium and is allowed to grow further for approximately 2 hours. IPTG is then added to 100 μM, and the culture is incubated with vigorous shaking for approximately 4 hours. The bacteria is harvested by centrifugation, and the cell pellet is washed, e.g., 3 times in PBS. The cells are resuspended in PBS+1% Triton X-100 and are sonicated in order to break the cell walls of the bacteria to release the protein from the cells. In the instances where expression of the antigen as a fusion protein to glutathion-S-transferase generates insoluble protein, solubilization can be achieved by adding urea, up to a final concentration of 8 M. Then, the fusion protein can be dialyzed against a buffer such as PBS. Other expression vectors suitable for the production of the IgE-containing polypeptide in bacteria have been described in (Krebber, A., S. Bornhauser, et al. (1997). “Reliable cloning of functional antibody variable domains from hybridomas and spleen cell repertoires employing a reengineered phage display system.” J Immunol Methods 201(l):35-55). Vectors useful for the production of IgE-containing polypeptide eukaryotic hosts have also been described (Hu, S., L. Shively et al. (1996). “Minibody: A novel engineered anticarcinoembryonic antigen antibody fragment (single-chain Fv-CH3) which exhibits rapid, high-level targeting of xenografts.” Cancer Res 56(13):3055-61).

If desired, IgE-containing polypeptides can be coupled to Keyhole Limpet Hemocyanin (KLH) (Sigma Chemical Co.) using conventional methods (See Burt et al., Molec. Immunol. 23:181-191 (1986) and Avrameas, ImmunocytochemistryI 6:43-52, (1969)). Such a coupling method can be carried out by glutaraldehyde crosslinking as follows, or using a heterobifunctional crosslinker such as ε-maleimidocaproic acid N-hydroxy-succinimide ester. A polypeptide (5 mg) in 1 ml of 0.1 N phosphate buffer (pH 7) is added to 10 mg KLH dissolved in 1 ml H₂O. One ml of glutaraldehyde (21 mM) in 0.1 N phosphate buffer at pH 7 is added dropwise, and the mixture is incubated at room temperature overnight with stirring. The solution then is dialyzed extensively against PBS, and can be stored at −20° C. until use. Alternatively, sulfo-MBS can be used instead of glutaraldehyde.

As stated above, the carrier includes a first attachment site, which binds to the second attachment site of the IgE-containing polypeptide. If desired, the first attachment site, included within the carrier, can be an amino acid sequence that specifically binds to antibodies. For example, the first attachment site may include protein A, or a portion of protein A that binds to a rodent (e.g., mouse or rat) CH2-CH3 domain of IgG (See Hellman, Eur. J. Immunol. 24:415-520 (1994) and Hellman et al., Nucl. Acids. Res. 10:6041(1982)). Alternatively, the first attachment site may include protein L, or a portion of protein L that binds to a variable region of an Ig light chain. If desired, the first attachment site can include a CH2-CH3 domain or an Ig light chain variable region, and the second attachment site includes protein A or protein L. In other embodiments, the first attachment site is a protein, a polypeptide, a peptide, a sugar, a polynucleotide, a natural or synthetic polymer, a metabolite or compound (e.g., biotin, fluorescein, retinol, digoxigenin, metal ions, phenylmethylsulfonyl fluoride), or a combination thereof, or a chemically reactive group thereof. Thus, the first attachment site may include an antigen, an antibody or antibody fragment, biotin, avidin, streptavidin, a ligand, a ligand-binding protein, an interacting leucine zipper polypeptide, an amino group, a chemical group reactive to an amino group; a carboxyl group, a chemical group reactive to a carboxyl group, a sulfhydryl group, a chemical group reactive to a sulfhydryl group, an engineered chemically reactive group, or a combination thereof.

A preferred embodiment of the invention utilizes a Sindbis virus as a carrier. The Sindbis virus RNA genome is packaged into a capsid protein that is surrounded by a lipid bilayer containing the E1, E2, and E3 proteins. The glycosylated portions of these glycoproteins are located on the outside of the lipid bilayer, and complexes of these proteins form “spikes” that project outward from the surface of the virus. In another preferred embodiment of the invention, the first attachment site is a JUN or FOS leucine zipper protein domain that is linked to an E1, E2, or E3 envelope protein. Alternatively, other envelope proteins may be utilized to provide a first attachment site in the carrier. In another embodiment of the invention, the first attachment site is a JUN or FOS leucine zipper protein domain that is linked to the Hepatitis B capsid (core) protein (HBcAg). A n exemplary JUN polypeptide has the following amino acid sequence: CGGRIARLEEKVKTLKAQ NSELASTANMLREQVAQLKQKVMNHVGC (SEQ ID NO:2). An exemplary FOS polypeptide has the following amino acid sequence: CGGLTDTLQAETDQVEDEKSALQTEIANLLKEKEKLEFILA AHGGC (SEQ ID NO:3). These sequences are derived from the transcription factors JUN and FOS, and each is flanked by a short sequence containing a cysteine residue on both sides. These sequences are known to interact with each other. The term “leucine zipper” is used to refer to the sequences depicted above or sequences essentially similar to the ones depicted above.

In order to simplify the generation of FOS fusion constructs, several vectors are disclosed. The vectors pAV1-4 were designed for the expression of FOS fusion proteins in E. coli; the vectors pAV5 and pAV6 were designed for the expression of FOS fusion proteins in eukaryotic cells. Properties of these vectors are briefly described:

pAV1: This vector was designed for the secretion of fusion proteins with FOS at the C-terminus into the E. coli periplasmic space. The gene of interest (g.o.i.) may be ligated into the StuI/NotI sites of the vector.

pAV2: This vector was designed for the secretion of fusion proteins with FOS at the N-terminus into the E. coli periplasmic space. The gene of interest can be ligated into the NotI/EcoRV (or NotI/HindIII) sites of the vector.

pAV3: This vector was designed for the cytoplasmic production of fusion proteins with FOS at the C-terminus in E. coli. The gene of interest (g.o.i.) may be ligated into the EcoRV/NotI sites of the vector.

pAV4: This vector is designed for the cytoplasmic production of fusion proteins with FOS at the N-terminus in E. coli. The gene of interest (g.o.i.) may be ligated into the NotI/EcoRV (or NotI/HindIII) sites of the vector. The N-terminal methionine residue is proteolytically removed upon protein synthesis (Hirel et al., Proc. Natl. Acad. Sci. USA 86:8247-8251 (1989)).

pAV5: This vector was designed for the eukaryotic production of fusion proteins with FOS at the C-terminus. The gene of interest (g.o.i.) may be inserted between the sequences coding for the hGH signal sequence and the FOS domain by ligation into the Eco47III/NotI sites of the vector. Alternatively, a gene containing its own signal sequence may be fused to the FOS coding region by ligation into the StuI/NotI sites.

pAV6: This vector was designed for the eukaryotic production of fusion proteins with FOS at the N-terminus. The gene of interest (g.o.i.) may be ligated into the NotI/StuI (or NotI/HindIII) sites of the vector.

Assembly of the ordered and repetitive array in the JUN/FOS embodiment can be done in the presence of a redox shuffle. E2-JUN viral particles are combined with a 240 fold molar excess of FOS-antigen or FOS-antigenic determinant for 10 hours at 4° C. Subsequently, the alphaviral particles are concentrated and purified by chromatography. As will be understood by those skilled in the art, the construction of a fusion protein may include the addition of certain genetic elements to facilitate production of the recombinant protein, e.g., E. coli regulatory elements for translation, or a eukaryotic signal sequence. Other genetic elements may be selected, depending on the specific needs of the practitioner.

In certain embodiments, the carrier used in compositions of the invention includes a Hepatitis B capsid (core) protein (HBcAg), or a fragment thereof, which, optionally, has been modified to eliminate or reduce the number of free cysteine residues, as described in copending non-provisional application 09/848,616; filed May 4, 2001; herein incorporated by reference. (See also Zhou et al. J. Virol. 66:5393-5398 (1992)). HBcAgs that have been modified to remove the naturally resident cysteine residues retain the ability to associate and form multimeric structures. The naturally resident cysteine residues can be deleted or substituted with another amino acid residue (e.g., a serine residue). The HBcAg is a protein generated by the processing of a Hepatitis B core antigen precursor protein. Various isotypes of the HBcAg have been identified. For example, an HBcAg protein having the amino acid sequence shown in SEQ ID NO:4 is generated by the processing of a 212 amino acid Hepatitis B core antigen precursor protein, resulting in the removal of 29 amino acids from the N-terminus. Similarly, an HBcAg protein having the amino acid sequence shown in SEQ ID NO:5 is generated by the processing of a 214 amino acid Hepatitis B core antigen precursor protein. The amino acid sequence shown in SEQ ID NO:5, as compared to the amino acid sequence shown in SEQ ID NO:4, contains a two amino acid insert at positions 152 and 153 in SEQ ID NO:5.

Further, the HBcAg variants used to prepare compositions of the invention will generally be variants which retain the ability to associate with other HBcAgs to form dimeric or multimeric structures that present ordered and repetitive antigen or antigenic determinant arrays.

Another preferred HBcAg polypeptide, HBcAg-Lys, is MDIDPYKEFG ATVELLSFLPSDFFPSVRDLLDTASALYREAIESPEHCSPHHTALRQAIL CWGELMTLATWVGTNLEDGGKGGSRDLVVSYVNTNMGLKIRQLLW FHISCLTFGRETVLEYLVSFGVWIRTPPAYRPPNAPILSTLPETTVV (SEQ ID NO:6). Another preferred HBcAg polypeptide, HBcAg-Lys-2cys-Mut, is MDIDPYKEFGATVELLSFLPSDFFPSVRDLLDTASALYREALESPEHSSP HHTALRQAILCWGELMTLATWVGTNLEDGGKGGSRDLVVSYVNTN MGLKIRQLLWFHISSLTFGRETVLEYLVSFGVWIRTPPAYRPPNAPILST LPETTVV (SEQ ID NO:7).

Preferably, compositions of the invention include an HBcAg from which the N-terminal leader sequence (e.g., the first 29 amino acid residues shown in SEQ ID NO:8) of the Hepatitis B core antigen precursor protein have been removed. If HBcAgs are produced under conditions under which processing does not occur, the HBcAgs generally are expressed in “processed” form. For example, bacterial systems, such as E. coli, generally do not remove the leader sequences of proteins which are normally expressed in eukaryotic cells. Thus, when an E. coli expression system is used to produce HBcAgs of the invention, these proteins will generally be expressed such that the N-terminal leader sequence of the Hepatitis B core antigen precursor protein is not present.

In some embodiments, compositions of the invention contain HBcAgs that have nucleic acid binding activity (e.g., which contain a naturally resident HBcAg nucleic acid binding domain). HBcAgs containing one or more nucleic acid binding domains are useful for preparing compositions having enhanced T-cell stimulatory activity.

In other embodiments, compositions of the invention will contain HBcAgs from which the C-terminal region (e.g., amino acid residues 145-185 or 150-185 of SEQ ID NO:8) has been removed, and which do not bind nucleic acids. Thus, additional modified HBcAgs suitable for use in the present invention include C-terminal truncation mutants. Suitable C-terminal truncation mutants include HBcAgs from which 1, 5, 10, 15, 20, 25, 30, 34, 35, 36, 37, 38, 39 40, 41, 42 or 48 amino acids have been removed.

HBcAgs suitable for use in the practice of the present invention also include N-terminal truncation mutants. Suitable N-terminal truncation mutants include modified HBcAgs from which 1, 2, 5, 7, 9, 10, 12, 14, 15, and 17 amino acids have been removed.

The invention also includes vaccine compositions in which the carrier is fused to an additional protein, e.g., a HBcAg/FOS fusion. Other examples of HBcAg fusion proteins suitable for use as carriers in compositions of the invention include fusion proteins in which an amino acid sequence has been added which aids in the formation and/or stabilization of HBcAg dimers and multimers. This additional amino acid sequence may be fused to either the N- or C-terminus of the HBcAg. One example, of such a fusion protein is a fusion of a HBcAg with the GCN4 helix region of Saccharomyces cerevisiae (GenBank Accession No. P03069, which is incorporated herein by reference).

HBcAg/src homology 3 (SH3) domain fusion proteins can also be used to prepare compositions of the invention. SH3 domains are relatively small domains found in a number of proteins which confer the ability to interact with specific proline-rich sequences in protein binding partners (see McPherson, Cell Signal 11:229-238 (1999)). HBcAg/SH3 fusion proteins can be used in several ways. First, the SH3 domain can form a first attachment site which interacts with a second attachment site. Similarly, a proline rich amino acid sequence could be added to the HBcAg and used as a first attachment site for an SH3 domain second attachment site. Second, the SH3 domain could associate with proline rich regions introduced into HBcAgs. Thus, SH3 domains and proline rich SH3 interaction sites could be inserted into either the same or different HBcAgs and used to form stabilized dimers and multimers.

A variety of host cells can be utilized to produce a viral carrier for use in the compositions of the invention. For example, Alphaviruses have a wide host range; Sindbis virus infects cultured mammalian, reptilian, and amphibian cells, as well as some insect cells (Clark, H., J. Natl. Cancer Inst. 51:645 (1973); Leake, C., J. Gen. Virol. 35:335 (1977); Stollar, V. in THE TOGAVIRUSES, R. W. Schlesinger, Ed., Academic Press, (1980), pp.583-621). BHK, COS, Vero, HEK 293 and CHO cells are particularly suitable because they can glycosylate heterologous proteins in a manner similar to human cells (Watson, E. et al., Glycobiology 4:227, (1994)), and they can be selected (Zang, M. et al., Bio/Technology 13:389 (1995)) or genetically engineered (Renner W. et al., Biotech. Bioeng. 4.476 (1995); Lee K. et al. Biotech. Bioeng. 50:336 (1996)) to grow in serum-free medium, as well as in suspension. HeLa cells can also be used. Other hosts, such as E. coli (Zlotnick, A., N. Cheng et al. (1996). “Dimorphism of hepatitis B virus capsids is strongly influenced by the C-terminus of the capsid protein.” Biochemistry 35(23):7412-21) or Yeast (Kniskern, P. J., A. Hagopian, et al. (1986). “Unusually high-level expression of a foreign gene (hepatitis B virus core antigen) in Saccharomyces cerevisiae.” Gene 46(1):135-41).

Vectors can be introduced into host cells by using conventional techniques manuals (see, e.g., Sambrook, J. et al., eds., MOLECULAR CLONING, A LABORATORY MANUAL, 2nd. edition, Cold Spring Harbor Laboratory Press, Cold Spring Harbor, N.Y. (1989), Chapter 9; Ausubel, F. et al., eds., CURRENT PROTOCOLS IN MOLECULAR BIOLOGY, John H. Wiley & Sons, Inc. (1997), Chapter 16). Examples of suitable methods include, without limitation, electroporation, DEAE-dextran mediated transfection, transfection, microinjection, cationic lipid-mediated transfection, transduction, scrape loading, ballistic introduction, and infection. Methods for introducing DNA sequences into host cells are discussed in U.S. Pat. No. 5,580,859.

If desired, packaged RNA sequences can be introduced to host cells by adding them to the culture medium. For example, the preparation of non-infective alphaviral particles is described in a number of sources, including “Sindbis Expression System,” Version C (Invitrogen Catalog No. K750-1).

When mammalian cells are used as recombinant host cells for the production of viral carriers, such cells can be cultured using standard techniques (see, e.g., Celis, J., ed., CELL BIOLOGY, Academic Press, 2^{nd} edition, (1998); Sambrook, J. et al., eds., MOLECULAR CLONING, A LABORATORY MANUAL, 2nd. edition, Cold Spring Harbor Laboratory Press, Cold Spring Harbor, N.Y. (1989); Ausubel, F. et al., eds., CURRENT PROTOCOLS IN MOLECULAR BIOLOGY, John H. Wiley & Sons, Inc. (1997); Freshney, R., CULTURE OF ANIMAL CELLS, Alan R. Liss, Inc. (1983)).

In general, the association between the attachment and second attachment sites will be determined by the characteristics of the respective molecules selected but will typically comprise at least one non-peptide bond. Depending upon the combination of the first and second attachment sites, the nature of the association may be covalent, ionic, hydrophobic, polar, or a combination thereof.

The invention provides novel compositions and methods for the construction of ordered and repetitive arrays of IgE-containing polypeptides. The conditions for the assembly of the ordered and repetitive arrays depend on the choice of the first and second attachment sites. Information relating to assembly of Alphaviral particles, for example, is well within the working knowledge of the practitioner, and numerous references exist to aid the practitioner (e.g., Sambrook, J. et al., eds., Molecular Cloning, A Laboratory Manual, 2nd. edition, Cold Spring Harbor Laboratory Press, Cold Spring Harbor, N.Y. (1989); Ausubel, F. et al., eds., Current Protocols in Molecular Biology, John H. Wiley & Sons, Inc. (1997); Celis, J., ed., Cell Biology, Academic Press, 2^{nd} edition, (1998); Harlow, E. and Lane, D., Antibodies: A Laboratory Manual, Cold Spring Harbor Laboratory, Cold Spring Harbor, N.Y. (1988), all of which are incorporated herein by reference).

In another embodiment of the invention, the coupling of the carrier to the IgE-containing polypeptide may be accomplished by chemical cross-linking. In a specific embodiment, the chemical agent is a heterobifunctional cross-linking agent such as ε-maleimidocaproic acid N-hydroxy-succinimide ester (Tanimori et al., J. Pharm. Dyn. 4:812 (1981); Fujiwara et al., J. Immunol. Meth. 45:195 (1981)), which contains (1) a N-hydroxy-succinimide ester group reactive with amino groups and (2) a maleimide group reactive with SH groups. Other hetero-bifunctional cross-linkers can be used in the present invention such as, by way of example, SMCC (Succinimidyl 4-[N-maleimidomethyl]-cyclohexane-1-carboxylate), SMPB (Succinimidyl 4-p-maleimidophenyl]-butyrate), (N-[γ-Maleimidobutylody]sulfosuccinimide ester), Sulfo-SMCC (Sulfosuccinimidyl 4 [N-maleimidomethyl]-cyclohexane-1-carboxylate), Succinimidyl-3-[bromoacetamido] propionate and SIAB (from the supplier Pierce) can also be used in making compositions of the invention.

A second attachment site of the IgE-containing polypeptide or a second attachment site of the carrier may be engineered to contain one or more lysine residues that will serve as a reactive moiety for the N-hydroxy-succinimide ester portion of the heterobifunctional cross-linking agent. Moreover, a second attachment site of the IgE-containing polypeptide or first attachment site of the carrier can be engineered to contain one or more cysteine residues that will serve as a reactive moiety for the maleimide portion of the heterobifunctional cross-linking agent.

In a first, preferred embodiment, the N-hydroxy-succinimide ester group is chemically coupled to a lysine residue of the carrier. Once chemically coupled to the lysine residue of the carrier, the maleimide group of the heterobifunctional cross-linking agent will be available to react with the SH group of a cysteine residue of a first attachment site of the IgE-containing polypeptide. Preparation of the carrier may require the engineering of a lysine residue into the carrier's attachment site so that it may be attached to the heterobifunctional cross-linking agent. Preparation of the IgE-containing polypeptide may require the engineering of a cysteine residue into the IgE-containing polypeptide at the second attachment site so that it may be reacted with the free maleimide on the cross-linking agent bound to the carrier.

In an alternatively preferred embodiment, the N-hydroxy-succinimide ester group is chemically coupled to a lysine residue of the IgE-containing polypeptide. Once chemically coupled to the lysine residue of the IgE-containing polypeptide, the maleimide group of the heterobifunctional cross-linking agent will be available to react with the SH group of a cysteine residue of an attachment site of the carrier. Preparation of the IgE-containing polypeptide may require the engineering of a lysine residue into the IgE-containing polypeptide's second attachment site so that it may be attached to the heterobifunctional cross-linking agent. Preparation of the carrier may require the engineering of a cysteine residue into the carrier's attachment site so that it may be reacted with the free maleimide on the cross-linking agent bound to the carrier.

Thus, in such an instance, the heterobifunctional cross-linking agent couples the carrier to the IgE-containing polypeptide via the first and second attachment site.

### Bacterial Pili

Bacterial pili can also be used as carriers in the compositions of the invention. Bacterial pili or fimbriae are filamentous surface organelles produced by a wide range of bacteria. These organelles mediate the attachment of bacteria to surface receptors of host cells and are required for the establishment of many bacterial infections like cystitis, pyelonephritis, new born meningitis and diarrhea.

Pili can be divided in different classes with respect to their receptor specificity (agglutination of blood cells from different species), their assembly pathway (extracellular nucleation, general secretion, chaperone/usher, alternate chaperone) and their morphological properties (thick, rigid pili; thin, flexible pili; atypical structures including capsule; curli; etc.). Examples of thick, rigid pili forming a right handed helix that are assembled via the so called chaperone/usher pathway and mediate adhesion to host glycoproteins include Type-1 pili, P-pili, S-pili, F1C-pili, and 987P-pili (for reviews on adhesive structures, their assembly and the associated diseases see Soto, G. E. & Hultgren, S. J., J. Bacteriol. 181:1059-1071 (1999); Bullitt & Makowski, Biophys. J. 74:623-632 (1998); Hung, D. L. & Hultgren, S. J., J. Struct, Biol. 124:201-220 (1998)).

Type-1 pili are long, filamentous polymeric protein structures on the surface of E. coli. They possess adhesive properties that allow for binding to mannose-containing receptors present on the surface of certain host tissues. Type-1 pili can be expressed by 70-80% of all E. coli isolates and a single E. coli cell can bear up to 500 pili. Type-1 pili reach a length of typically 0.2 to 2 μM with an average number of 1000 protein subunits that associate to a right-handed helix with 3.125 subunits per turn with a diameter of 6 to 7 nm and a central hole of 2.0 to 2.5 nm.

The main Type-1 pilus component, FimA, which represents 98% of the total pilus protein, is a 15.8 kDa protein. The minor pilus components FimF, FimG and FimH are incorporated at the tip and in regular distances along the pilus shaft (Klemm, P. & Krogfelt, K. A., “Type I fimbriae of Escherichia coli,” in: Fimbriae. Klemm, P. (ed.), CRC Press Inc., (1994) pp. 9-26). FimH, a 29.1 kDa protein, was shown to be the mannose-binding adhesin of Type-1 pili (Krogfelt, K. A., et al., Infect. Immun. 58:1995-1998 (1990); Klemm, P., et al., Mol. Microbiol. 4:553-560 (1990); Hanson, M. S. & Brinton, C. C. J., Nature 17:265-268 (1988)), and its incorporation is probably facilitated by FimG and FimF (Klemm, P. & Christiansen, G., Mol. Gen. Genetics 208:439-445 (1987); Russell, P. W. & Orndorff, P. E., J. Bacteriol. 174:5923-5935 (1992)). The order of major and minor components in the individual mature pili is very similar, indicating a highly ordered assembly process (Soto, G. E. & Hultgren, S. J., J. Bacteriol. 181:1059-1071 (1999)).

P-pili of E. coli are of very similar architecture, have a diameter of 6.8 nm, an axial hole of 1.5 nm and 3.28 subunits per turn (Bullitt & Makowski, Biophys. J. 74:623-632 (1998)). The 16.6 kDa PapA is the main component of this pilus type and shows 36% sequence identity and 59% similarity to FimA (see Table 1). As in Type-1 pili the 36.0 kDa P-pilus adhesin PapG and specialized adapter proteins make up only a tiny fraction of total pilus protein. The most obvious difference to Type-1 pili is the absence of the adhesin as an integral part of the pilus rod, and its exclusive localization in the tip fibrillium that is connected to the pilus rod via specialized adapter proteins that Type-1 pili lack (Hultgren, S. J., et al., Cell 73:887-901 (1993)).

P-pili and Type-1 pili are encoded by single gene clusters on the E. coli chromosome of approximately 10 kb (Klemm, P. & Krogfelt, K. A., “Type I fimbriae of Escherichia coli,” in: Fimbriae. Klemm, P. (ed.), CRC Press Inc., (1994) pp. 9-26; Orndorff, P. E. & Falkow, S., J. Bacteriol. 160:61-66 (1984)). A total of nine genes are found in the Type-1 pilus gene cluster, and 11 genes in the P-pilus cluster (Hultgren, S. J., et al., Adv. Prot. Chem. 44:99-123 (1993)). Both clusters are organized quite similarly. The assembly platform in the outer bacterial membrane to which the mature pilus is anchored is encoded by the fimD gene (Klemm, P. & Christiansen, G., Mol. Gen, Genetics 220:334-338 (1990)). The three minor components of the Type-1 pili, FimF, FimG and FimH are encoded by the last three genes of the cluster (Klemm, P. & Christiansen, G., Mol Gen. Genetics 208:439-445 (1987)). Apart from fimB and fimE, all genes encode precursor proteins for secretion into the periplasm via the sec-pathway.

Type-1 pili as well as P-pili are to 98% made of a single or main structural subunit termed FimA and PapA, respectively. Both proteins have a size of ˜5.5 kDa. The additional minor components encoded in the pilus gene clusters are very similar.

In various embodiments, a bacterial pilin, a subportion of a bacterial pilin, or a fusion protein which contains a bacterial pilin or subportion thereof is used to prepare carriers for use in compositions of the invention. Examples of pilin proteins include pilins produced by Escherichia coli, Haemophilus influenzae, Neisseria meningitidis, Neisseria gonorrhoeae, Caulobacter crescentus, Pseudomonas stutzeri, and Pseudomonas aeruginosa. The amino acid sequences of pilin proteins suitable for use with the present invention include those set out in GenBank reports AJ000636, AJ132364, AF229646, AF051814, and AF051815, the entire disclosures of which are incorporated herein by reference. One exemplary pilin protein suitable for use in the present invention is the P-pilin of E. coli (GenBank report AF237482). An example of a Type-1 E. coli pilin suitable for use with the invention is a pilin having the amino acid sequence set out in GenBank report P04128. The entire disclosures of these GenBank reports are incorporated herein by reference.

Bacterial pilins or pilin subportions suitable for use in the practice of the present invention will generally be able to associate to form soluble carriers. Methods for preparing pili and pilus-like structures in vitro are known in the art. Bullitt et al., Proc. Natl. Acad. Sci. USA 93:12890-12895 (1996), for example, describe the in vitro reconstitution of E. coli P-pili subunits. Further, Eshdat et al., J. Bacteriol. 148:308-3 14 (1981) describe methods suitable for dissociating Type-1 pili of E. coli and the reconstitution of both pilin dimers and pili. In brief, these methods are as follows: pili are dissociated by incubation at 37° C. in saturated guanidine hydrochloride. Pilin proteins are then purified by chromatography, after which pilin dimers are formed by dialysis against 5 mM tris(hydroxymethyl)aminomethane hydrochloride (pH 8.0). Eshdat et al. also found that pilin dimers reassemble to form pili upon dialysis against the 5 mM tris(hydroxymethyl)aminomethane (pH 8.0) containing 5 mM MgCl₂.

By using conventional genetic engineering and protein modification methods, pilin proteins may be modified to contain a first attachment site to which an IgE-containing polypeptide is coupled through a second attachment site. Alternatively, IgE-combining polypeptides can be directly linked through a first attachment site to amino acid residues which are naturally resident in pilin proteins. These modified pilin proteins may then be used in compositions of the invention.

Bacterial pilin proteins used to prepare compositions of the invention may be modified in a manner similar to that described herein for HBcAg. For example, cysteine and lysine residues may be either deleted or substituted with other amino acid residues and attachment sites may be added to these proteins. These pilin proteins may then be reassembled using methods, for example, similar to those described above.

In another embodiment, pili or pilus-like structures are harvested from bacteria (e.g., E. coli) and used to form compositions of the invention. One example of pili suitable for preparing compositions is the Type-1 pilus of E. coli, which is formed from pilin monomers having the amino acid sequence set out in SEQ ID NO:8.

A number of methods for harvesting bacterial pili are known in the art. Bullitt and Makowski (Biophys. J. 74:623-632 (1998)), for example, describe a pilus purification method for harvesting P-pili from E. coli. According to this method, pili are sheared from hyperpiliated E. coli containing a P-pilus plasmid and purified by cycles of solubilization and MgCl₂ (1.0 M) precipitation.

Once harvested, pili or pilus-like structures may be modified in a variety of ways. For example, a first attachment site can be added to the pili to which antigens or antigen determinants may be attached through a first attachment site. In other words, bacterial pili or pilus-like structures can be harvested and modified to form carriers. Pili or pilus-like structures may also be modified by the direct attachment of IgE-containing polypeptides. For example, IgE-containing polypeptides can be linked through a heterobifunctional crosslinker to resident cysteine residues or lysine residues of bacterial pilin proteins.

When structures which are naturally synthesized by organisms (e.g., pili) are used to prepare compositions of the invention, it will often be advantageous to genetically engineer these organisms so that they produce structures having desirable characteristics. For example, when Type-1 pili of E. coli are used, the E. coli from which these pili are harvested may be modified so as to produce structures with specific characteristics. Examples of possible modifications of pilin proteins include the insertion of one or more lysine or cysteine residues, the deletion or substitution of one or more of the naturally resident lysine residues, and the deletion or substitution of one or more naturally resident cysteine residues.

Further, additional modifications can be made to pilin genes which result in the expression products containing a first attachment site other than a lysine residue (e. g., a FOS or JUN domain). Of course, suitable attachment sites do not prevent pilin proteins from forming pili or pilus-like structures suitable for use in compositions of the invention.

Pilin genes which naturally reside in bacterial cells can be modified (e.g. by homologous recombination), or pilin genes with particular characteristics can be inserted into these cells. For example, pilin genes could be introduced into bacterial cells as a component of either a replicable cloning vector or a vector which inserts into the bacterial chromosome. The inserted pilin genes may also be linked to expression regulatory control sequences (e.g., a lac operator).

In most instances, the pili or pilus-like structures used in compositions of the invention will be composed of a single type of a pilin subunit. Pili or pilus-like structures composed of identical subunits will generally be used because they are expected to form structures which present highly ordered and repetitive arrays of the IgE-containing polypeptide. However, the compositions of the invention also include pili or pilus-like structures formed from heterogenous pilin subunits. The pilin subunits which form these pili or pilus-like structures can be expressed from genes naturally resident in the bacterial cell or may be introduced into the cells. When a naturally resident pilin gene and an introduced gene are both expressed in a cell which forms pili or pilus-like structures, the result will generally be structures formed from a mixture of these pilin proteins. Further, when two or more pilin genes are expressed in a bacterial cell, the relative expression of each pilin gene will typically be the factor which determines the ratio of the different pilin subunits in the pili or pilus-like structures.

When pili or pilus-like structures having a particular composition of mixed pilin subunits is desired, the expression of at least one of the pilin genes can be regulated by a heterologous, inducible promoter. Such promoters, as well as other genetic elements, can be used to regulate the relative amounts of different pilin subunits produced in the bacterial cell and, hence, the composition of the pili or pilus-like structures, if desired.

In addition, while in various embodiments the IgE-containing polypeptides will be coupled to bacterial pili or pilus-like structures by a bond which is not a peptide bond, bacterial cells which produce pili or pilus-like structures used inthe compositions of the invention can be genetically engineered to generate pilin proteins which are fused to an IgE-containing polypeptide. Such fusion proteins which form pili or pilus-like structures are suitable for use in compositions of the invention. Thus, IgE-containing polypeptides may be attached to pilin proteins by the expression of pilin/IgE fusion proteins. IgE-containing polypeptides may also be attached to bacterial pili, pilus-like structures, or pilin proteins through non-peptide bonds.

### Pharmaceutical Formulations

Compositions of the invention can be prepared for storage as lyophilized formulations or aqueous solutions by mixing the compositions with optional “pharmaceutically-acceptable” excipients typically employed in the art. For example, buffering agents, stabilizing agents, preservatives, isotonifiers, non-ionic detergents, antioxidants and other miscellaneous additives can be used. (See Remington's Pharmaceutical Sciences, 16th edition, A. Osol, ed. (1980)). Such additives must be nontoxic to the recipients at the dosages and concentrations employed.

In general, compositions of the invention may contain salts, buffers, adjuvants, or other substances which are desirable for improving the efficacy of the composition. Examples of materials suitable for use in preparing pharmaceutical compositions are provided in numerous sources including Remington's Pharmaceutical Sciences (Osol, A, ed., Mack Publishing Co., (1980)). Compositions of the invention are said to be “pharmacologically acceptable” if their administration can be tolerated by a recipient individual. Further, the compositions of the invention will be administered in a “therapeutically effective amount” (i.e., an amount that produces a desired physiological effect). The compositions of the present invention may be administered by various methods known in the art, but will normally be administered by injection, infusion, inhalation, oral administration, or other suitable methods. The compositions may also be administered intramuscularly, intravenously, or subcutaneously. Components of compositions for administration include sterile aqueous (e.g., saline) or non-aqueous solutions and suspensions. Examples of non-aqueous solvents are propylene glycol, polyethylene glycol, vegetable oils such as olive oil, and injectable organic esters such as ethyl oleate. Carriers or occlusive dressings can be used to increase skin permeability and enhance absorption.

Buffering agents help to maintain the pH in the range which approximates physiological conditions. They are preferably present at concentration ranging from about 2 mM to about 50 mM. Suitable buffering agents for use with the present invention include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, there may be mentioned phosphate buffers, histidine buffers and trimethylamine salts such as Tris.

Preservatives can be added to retard microbial growth, and are added in amounts ranging from 0.2%-1% (w/v). Suitable preservatives for use with the present invention include, without limitation, phenol, benzyl alcohol, meta-cresol, methyl paraben, propyl paraben, octadecyldimethylbenzyl ammonium chloride, benzalconium halides (e.g., chloride, bromide, iodide), hexamethonium chloride, alkyl parabens such as methyl or propyl paraben, catechol, resorcinol, cyclohexanol, and 3-pentanol.

Isotonifiers sometimes known as “stabilizers” can be present to ensure isotonicity of liquid compositions of the present invention and include polhydric sugar alcohols, e.g., trihydric or higher sugar alcohols, such as glycerin, erythritol, arabitol, xylitol, sorbitol and mannitol. Polyhydric alcohols can be present in an amount between 0.1% to 25% by weight, preferably 1% to 5% taking into account the relative amounts of the other ingredients.

Stabilizers include a broad category of excipients which can range in function from a bulking agent to an additive which solubilizes the therapeutic composition or helps to prevent denaturation or adherence to the container wall. Examples of typical stabilizers include polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thio sulfate; low molecular weight polypeptides (i.e. <10 residues); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trisaccacharides such as raffinose; polysaccharides such as dextran. Stabilizers are present in the range from 0.1 to 10,000 (wt/wt).

Non-ionic surfactants or detergents (also known as “wetting agents”) can be included to help solubilize the therapeutic composition as well as to protect the therapeutic composition against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), Pluronic polyols, polyoxyethylene sorbitan monoethers (Tween-20, Tween-80, etc.). Non-ionic surfactants are present in a range of about 0.05 mg/ml to about 1.0 mg/ml, preferably about 0.07 mg/ml to about 0.2 mg/ml.

Additional miscellaneous excipients include bulking agents, (e.g. starch), chelating agents (e.g. EDTA), antioxidants (e.g., ascorbic acid, methionine, vitamin E), and cosolvents. If desired, the compositions of the invention may also be entrapped in microcapsule prepared, for example, by coascervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, A. Osal, ed. (1980). The formulations to be used for in vivo administration should be sterile. This is readily accomplished, for example, by filtration through sterile filtration membranes.

Sustained-release preparations may be prepared if desired. Suitable examples of sustained-release preparations include semi-permeable matrices of solid hydrophobic polymers containing the compositions of the invention, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT (injectable microspheres composed of lactic acid-glycolic acid copolymer and leuprolide acetate). While polymers such as ethylene-vinyl acetate and lactic acid-glycolic acid enable release of molecules for over 100 days, certain hydrogels release proteins for shorter time periods.

The amount of the composition of the invention which will be effective in the treatment of a particular disorder or condition will depend on the nature of the disorder or condition, and can be determined by standard clinical techniques. Where possible, it is desirable to determine the dose-response curve and the pharmaceutical compositions of the invention first in vitro, and then in useful animal model systems prior to testing in humans.

It is contemplated that the compositions of the invention will be used to inhibit or prevent an IgE-mediated disorder in a mammal (e.g., a human). As used herein, the term “IgE-mediated disorder” means a condition or disease which is characterized by the overproduction of, and/or hypersensitivity to, immunoglobulin IgE. Specifically it includes conditions associated with anaphylactic hypersensitivity and atopic allergies, including for example: asthma, allergic rhinitis and conjunctivitis (hay fever), eczema, urticaria, and food allergies. Anaphylactic shock, usually caused by bee or snake stings, insect bites or parental medication, is also encompassed by this term. Typical substances causing allergies include: grass, ragweed, birch or mountain cedar pollens, house dust, mites, animal danders, mold, insect venom or drugs (e.g., penicillin). Treatment with the compositions of the invention should be beneficial not only before, but also after, the onset of allergic conditions.

In one embodiment, the composition is administered to a non-human mammal for the purposes of obtaining preclinical data, for example. Exemplary non-human mammals to be treated include non-human primates, dogs, cats, rodents and other mammals in which preclinical studies typically are performed. Such mammals may be established animal models for a disorder to be treated with the composition or may be used to study toxicity of the composition. Alternatively, the composition may be used to treat the animal suffering from an allergic disease. In each of these embodiments, dose escalation studies may be performed on the mammal.

The composition of the invention is administered by any suitable means, including parenteral, subcutaneous, intraperitoneal, intrapulmonary, and intranasal. Parenteral infusions include intramuscular, intravenous, intraarterial, intraperitoneal, or subcutaneous administration.

For the prevention or treatment of IgE-mediated disorders, the optimal dosage of the composition will depend on the type of disorder to be treated, the severity and course of the disorder, whether the composition is administered for preventive or therapeutic purposes, previous therapy, the patient's clinical history and response to the antibody mutant, and the discretion of the attending physician. The compositions of the invention are suitably administered to the patient at one time or over a series of treatments.

Depending on the type and severity of the disorder, one or several doses of about 1 μg to about 5 mg of the composition is administered to the patient. For repeated administrations over several days or longer, depending on the condition, the treatment is sustained until a desired suppression of symptoms of the disorder occurs. However, other dosage regimens may be useful. The progress of this therapy is easily monitored by conventional techniques and assays. For example, efficacy can be assessed by detecting decreased levels of serum IgE, decreased binding of IgE to mast cells, or decreased histamine release, for example, using conventional method. An amelioration of the symptoms of the IgE-mediated disorder, e.g., sneezing, watery eyes, runny nose, and/or itching, also provides an indication of the efficacy of the treatment. The composition will be formulated, dosed and administered in a manner consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The composition need not be, but is optionally, formulated with one or more agents currently used to prevent or treat the disorder in question. These are generally used in the same dosages and with administration routes as described above.

### EXAMPLES

### Construction of the pAV Vector Series for Expression of FOS Fusion Proteins

A versatile vector system was constructed that allows cytoplasmic production or secretion of N- or C-terminal FOS fusion proteins in bacteria or production of N- or C-terminal FOS fusion proteins in eukaryotic cells. The vectors pAV1-pAV4 which were designed for production of FOS fusion proteins in E. coli, encompass the DNA cassettes listed below, which contain the following genetic elements arranged in different orders: (a) a strong ribosome binding site and 5′-untranslated region derived from the E. coli ompA gene (aggaggtaaaaaacg) (SEQ ID NO:9); (b) a sequence encoding the signal peptide of E. coli outer membrane protein OmpA (MKKTAIAIAVALAGFATVAQA) (SEQ ID NO:10); (c) a sequence coding for the FOS dimerization domain flanked on both sides by two glycine residues and a cystine residue (CGGLTDTLQAETDQVEDEKSALQTEIANLLKEKEKLEFILAAHGGC) (SEQ ID NO:3); and (d) a region encoding a short peptidic linker AAASGG (SEQ ID NO:11) or GGSAAA (SEQ ID NO:12)) connecting the protein of interest to the FOS dimerization domain. Relevant coding regions are given in upper case letters. The arrangement of restriction cleavage sites allows easy construction of FOS fusion genes with or without a signal sequence. The cassettes are cloned into the EcoRI/HindIII restriction sites of expression vector pKK223-3 (Pharmacia) for expression of the fusion genes under control of the strong tac promoter.

### pAV1

This vector was designed for the secretion of fusion proteins with FOS at the C-terminus into the E. coli periplasmic space. The gene of interest may be ligated into the StuI/NotI sites of the vector.

EcoRI 31/11
gaa ttc agg agg taa aaa acg ATG AAA AAG ACA GCT ATC GCG ATT GCA GTG GCA CTG GCT
M K K T A I A I A V A L A
61/21 StuI NotI
GGT TTC GCT ACC GTA GCG CAG GCC tgg gtg ggg GCG GCC GCT TCT GGT GGT TGC GGT GGT
G F A T V A Q A (goi) A A A S G G C G G
121/41 151/51
CTG ACC CAC ACC CTG CAG GCG GAA ACC GAC CAG GTG GAA GAC GAA AAA TCC GCG CTG CAA
L T D T L Q A E T D Q V E D E K S A L Q
181/61 211/71
ACC GAA ATC GCG AAC CTG CTG AAA GAA AAA GAA AAG CTG GAG TTC ATC CTG GCG GCA CAC
T E I A N L L K E K E K L E F I L A A H
241/81 HindIII
GGT GGT TGC taa gct t (SEQ ID NO: 13)
G G C * A (SEQ ID NOs: 1O and 14)

### pAV2

This vector was designed for the secretion of fusion proteins with FOS at the N-terminus into the E. coli periplasmic space. The gene of interest ligated into the NotI/EcoRV (or NotI/HindIII) sites of the vector.

```
EcoRI 31/11
gaa ttc agg agg taa aaa acg ATG AAA AAG ACA GCT ATC GCG ATT GCA GTG GCA CTG GCT
                                                        M K K T A I A I A V A L A
61/21 StuI 91/31
GGT TTC GCT ACC GTA GCG CAG GCC TGC GGT GGT CTG ACC GAC ACC CTG CAG GCG GAA ACC
G F A T V A Q A C G G L T D T L Q A E T
121/41 151/51
GAC CAG GTG GAA GAC GAA AAA TCC GCG CTG CAA ACC GAA ATC GCG AAC CTG CTG AAA GAA
D Q V E D E K S A L Q T E I A N L L K E
181/61 211/71 NotI
AAA GAA AAG CTG GAG TTC ATC CTG GCG GCA CAC GGT GGT TGC GGT GGT TCT GCG GCC GCT
K E K L E F I L A A H G G C G G S A A A
241/81 ECoRV HindIII
ggg tgt ggg gat atc aag ctt (SEQ ID NO: 15)
    (goi) (SEQ ID NO: 16)
```

### pAV3

This vector was designed for the cytoplasmic production of fusion proteins with FOS at the C-terminus in E. coli. The gene of interest may be ligated into the EcoRV/NotI sites of the vector.

EcoRI EcoRV NotI
gaa ttc agg agg taa aaa gat atc ggg tgt ggg GCG GCC GCT TCT GGT GGT TGC GGT GGT
(goi) A A A S G G C G G
61/21 91/31
CTG ACC GAC ACV CTG CAG GCG GAA ACC GAC CAG GTG GAA GAC GAA AAA TCC GCG CTG CAA
L T D T L Q A E T D Q V E D E K S A L Q
121/41 151/51
ACC GAA ATC GCG AAC CTG CTG AAA GAA AAA GAA AAG CTG GAG TTC ATC CTG GCG GCA CAC
T E I A N L L K E K E K L E F I L A A H
181/61HindIII
GGT GGT TGC taa gct t (SEQ ID NO: 17)
G G C * (SEQ ID NO: 14)

### pAV4

This vector is designed for the cytoplasmic production of fusion proteins with FOS at the N-terminus in E. coli. The gene of interest may be ligated into the NotI/EcoRV (or NotI/HindIII) sites of the vector. The N-terminal methionine residue is proteolytically removed upon protein synthesis (Hirel et al., Proc. Natl. Acad. Sci. USA 86:8247-8251 (1989)).

EcoRI 31/11
gaa ttc agg agg taa aaa acg ATG GCT TGC GGT GGT CTG ACC GAC ACC CTG CAG GCG GAA
E F R R * K T M A C G G L T D T L Q A E
61/21 91/31
ACC GAC CAG GTG GAA GAC GAA AAA TCC GCC CTG CAA ACC GAA ATC GCG AAC CTG CTG AAA
T D Q V E D E K S A L Q T E I A N L L K
121/41 151/51 NotI
GAA AAA GAA AAG CTG GAG TTC ATC CTG GCG GCA CAC GGT GGT TGC GGT GGT TCT GCG GCC
E K E K L E F I L A A H G G C G G S A A
181/61 ECoRV HindIII
GCT ggg tgt ggg gat atc aag ctt (SEQ ID NO: 18)
A (goi) (SEQ ID NOs: 19 and 20)

The vectors pAV5 and pAV6, which are designed for eukaryotic production of FOS fusion proteins, encompass the following genetic elements arranged in different orders: (a) a region coding for the leader peptide of human growth hormone (MATGSRTSLLLAFGLLCLPWLQEGSA) (SEQ ID NO:21); (b) a sequence coding for the FOS dimerization domain flanked on both sides by two glycine residues and a cysteine residue

(CGGLTDTLQAETDQVEDEKSALQTEIANLLKEKEKLEFILAAHGGC)
(SEQ ID NO: 3); and

(c) a region encoding a short peptidic linker (AAASGG (SEQ ID NO:11) or GGSAAA (SEQ ID NO:12)) connecting the protein of interest to the FOS dimerization domain. Relevant coding regions are given in upper case letters. The arrangement of restriction cleavage sites allows easy construction of FOS fusion genes. The cassettes are cloned into the EcoRI/HindIII restriction sites of the expression vector pMPSVEH (Artelt et al., Gene 68:213-219 (1988)).

### pAV5

This vector is designed for the eukaryotic production of fusion proteins with FOS at the C-terminus. The gene of interest may be inserted between the sequences coding for the hGH signal sequence and the FOS domain by ligation into the Eco47III/NotI sites of the vector. Alternatively, a gene containing its own signal sequence may be fused to the FOS coding region by ligation into the StuI/NotI sites.

EcoRI```StuI````````````````````````````31/11
gaa`ttc`agg`cct`ATG GCT ACA GGC TCC CGG ACG TCC CTG CTC CTG GCT TTT GGC CTG CTC
````````````````M```A```T```G```S```R```T```S```L```L```L```A```F```G```L```L
61/21```````````````````````````Eco47111````````````NotI
TGC CTG CCC TGG CTT CAA GAG GGC AGC`GCT`ggg tgt ggg GCG`GCC`GCT`TCT GGT GGT TGC
C```L```P```W```L```Q```E```G```S```A`````(goi)`````A```A```A```S```G```G```C
121/41``````````````````````````````````151/51
GGT GGT CTG ACC GAC ACC CTG CAG GCG GAA ACC GAC CAG GTG GAA GAC GAA AAA TCC GCG
G```G```L```T```D```T```L```Q```A```E```T```D```Q```V```E```D```E```K```S```E
181/61``````````````````````````````````211/71
CTG CAA ACC GAA ATC GCG AAC CTG CTG AAA GAA AAA GAA AAG CTG GAG TTC ATC CTG GCG
L```Q```T```E```I```A```N```L```L```K```E```K```E```K```L```E```F```I```L```A
241/81`````````````HindIII
GCA CAC GGT GGT TGC taa`gct`t`````````(SEQ ID NO: 22)
A```H```G```G```C```*`````````````````(SEQ ID NO: 14)

### pAV6

This vector is designed for the eukaryotic production of fusion proteins with FOS at the N-terminus. The gene of interest may be ligated into the NotI/StuI (or NotI/HindIII) sites of the vector.

```
EcoRI 31/l1
gaa ttc ATG GCT ACA GGC TCC CGG ACG TCC CTG CTC CTG GCT TTT GGC CTG GTC TGC CTG
                M A T G S R T S L L L A F G L L C L
61/21 Eco47III 91/31
CCC TGG CTT CAA GAG GGC AGC GCT TGC GGT GGT CTG ACC GAG ACC CTG CAG GCG GAA ACC
P W L Q E G S A C G G L T D T L Q A E T
121/41 151/51
GAC CAG GTG GAA GAC GAA AAA TCC GCG CTG CAA ACC GAA ATC GCG AAC CTG CTG AAA GAA
D Q V E D E K S A L Q T E I A N L L K E
181/61 211/71 NotI
AAA GAA AAG CTG GAG TTC ATC CTG GCG GCA CAC GGT GGT TGC GGT GGT TCT GCG GCC GCT
K E K L E F I L A A H G G C G G S A A A
241/81 StuI HindIII
ggg tgt ggg agg cct aag ctt (SEQ ID NO: 23)
    (goi) (SEQ ID NO: 24)
```

### Construction of Expression Vectors pAV1-pAV6

The following oligonucleotides have been synthesized for construction of expression vectors pAV1-pAV6:

| | |
|---|---|
| FOS-FOR1: | |
| CCTGGGTGGGGGCGGCCGCTTCTGGTGGTTGCGGTGGTCTGACC | (SEQ ID NO: 25); |
| FOS-FOR2: | |
| GGTGGGAATTCAGGAGGTAAAAAGATATCGGGTGTGGGGCGGCC | (SEQ ID NO: 26); |
| FOS-FOR3: | |
| GGTGGGAATTCAGGAGGTAAAAAACGATGGCTTGCGGTGGTCTGACC | (SEQ ID NO: 27); |
| FOS-FOR4: | |
| GCTTGCGGTGGTCTGACC | (SEQ ID NO: 28); |
| FOS-REV1: | |
| CCACCAAGCTTAGCAACCACCGTGTGC | (SEQ ID NO: 29); |
| FOS-REV2: | |
| CCACCAAGCTTGATATCCCCACACCCAGCGGCCGCAGAACCACCGC | (SEQ ID NO: 30); |
| AACCACCG | |
| FOS-REV3: | |
| CCACCAAGCTTAGGCCTCCCACACCCAGCGGC | (SEQ ID NO: 31); |
| OmpA-FOR1: | |
| GGTGGGAATTCAGGAGGTAAAAAACGATG | (SEQ ID NO: 32); |
| hGH-FORl: | |
| GGTGGGAATTCAGGCCTATGGCTACAGGCTCC | (SEQ ID NO: 33); and |
| hGH-FOR2: | |
| GGTGGGAATTCATGGCTACAGGCTCCC | (SEQ ID NO: 34). |

For the construction of vector pAV2, the regions coding for the OmpA signal sequence and the FOS domain were amplified from the ompA-FOS-hGH fusion gene in vector pKK223-3 using the primer pair OmpA-FOR1/FOS-REV2. The PCR product was digested with EcoRI/HindIII and ligated into the same sites of vector pKK223-3 (Pharmacia).

For the construction of vector pAV1, the FOS coding region was amplified from the ompA-FOS-hGH fusion gene in vector pKK223-3 using the primer pair FOS-FOR1/FOS-REV1. The PCR product was digested with HindIII and ligated into StuI/HindIII digested vector pAV2.

For the construction of vector pAV3, the region coding for the FOS domain was amplified from vector pAV1 using the primer pair FOS-FOR2/FOS-REV1. The PCR product was digested with EcoRI/HindIII and ligated into the same sites of the vector pKK223-3 (Pharmacia).

For the construction of vector pAV4, the region coding for the FOS domain was amplified from the ompA-FOS-hGH fusion gene in vector pKK223-3 using the primer pair FOS-FOR3/FOS-REV2. The PCR product was digested with EcoRI/HindIII and ligated into the same sites of the vector pKK223-3 (Pharmacia).

For the construction of vector pAV5, the region coding for the hGH signal sequence is amplified from the hGH-FOS-hGH fusion gene in vector pSINrep5 using the primer pair hGH-FOR1/hGHREV1. The PCR product is digested with EcoRI/NotI and ligated into the same sites of the vector pAV1. The resulting cassette encoding the hGH signal sequence and the FOS domain is then isolated by EcoRI/HindIII digestion and cloned into vector pMPSVEH (Artelt et al., Gene 68:213-219 (1988)) digested with the same enzymes.

For the construction of vector pAV6, the FOS coding region is amplified from vector pAV2 using the primer pair FOS-FOR4/FOSREV3. The PCR product is digested with HindIII and cloned into Eco47III/HindIII cleaved vector pAV5. The entire cassette encoding the hGH signal sequence and the FOS domain is then reamplified from the resulting vector using the primer pair hGH-FOR2/FOSREV3, cleaved with EcoRI/HindIII and ligated into vector pMPSVEH (Artelt et al., Gene 68:213-219 (1988)) cleaved with the same enzymes.

### Preparation of Alpha Viral Particles

Viral particles can be concentrated using Millipore Ultrafree Centrifugal Filter Devices with a molecular weight cut-off of 100 kD according to the protocol supplied by the manufacturer. Alternatively, viral particles can be concentrated by sucrose gradient centrifugation as described in the instruction manual of the Sindbis Expression System (Invitrogen, San Diego, Calif.). The pH of the virus suspension is adjusted to 7.5 and viral particles are incubated in the presence of 2-10 mM DTT for several hours. Viral particles can be purified from contaminating protein on a Sephacryl S-300 column (Pharmacia) (viral particles elute with the void volume) in an appropriate buffer.

Purified virus particles are incubated with at least 240 fold molar excess of FOS-antigen fusion protein in an appropriate buffer (pH 7.5-8.5) in the presence of a redox shuffle (oxidized glutathione/reduced glutathione; cystine/cysteine) for at least 10 hours at 4° C. After concentration of the particles using a Millipore Ultrafree Centrifugal Filter Device with a molecular weight cut-off of 100 kD, the mixture is passed through a Sephacryl S-300 gel filtration column (Pharmacia). Viral particles are eluted with the void volume. Other methods for producing viral particles also can be used.

### Covalent Coupling of FOS to JUN

To demonstrate binding of a FOS-containing protein to HBcAg-JUN particles, human growth hormone (hGH) fused at its carboxyl terminus to the FOS helix was used as a model protein (hGH-FOS). HBcAg-JUN particles were mixed with partially purified hGH-FOS and incubated for 4 hours at 4° C. to allow binding of the proteins. The mixture was then dialyzed overnight against a 3000-fold volume of dialysis buffer (150 mM NaCl, 10 mM Tris-HCl solution, pH 8.0) in order to remove DTT present in both the HBcAg-JUN solution and the hGH-FOS solution and thereby allow covalent coupling of the proteins through the establishment of disulfide bonds. As controls, the HBcAg-JUN and the hGH-FOS solutions were also dialyzed against dialysis buffer. Samples from all three dialyzed protein solutions were analyzed by SDS-PAGE under non-reducing conditions. Coupling of hGH-FOS to HBcAg-JUN was detected in an anti-hGH immunoblot. hGH-FOS bound to HBcAg-JUN should migrate with an apparent molecular mass of approximately 53 kDa, while unbound HGH-FOS migrates with an apparent molecular mass of 1 kDa. The dialysate was analyzed by SDS-PAGE in the absence of reducing agent and in the presence of reducing agent and detected by Coomassie staining. As a control, hGH-FOS that had not been mixed with capsid particles was also loaded on the gel in the presence of reducing agent. A shift of hGH-FOS to a molecular mass of approximately 53 kDa was observed in the presence of HBcAg-JUN capsid protein, indicating that efficient binding of hGH-FOS to HBcAg-JUN had taken place.

### Chemical Coupling of FLAG Peptide of HBcAg-Lys using the Heterobifunctional Cross-linker SPDP

Synthetic FLAG peptide with a Cysteine residue at its amino terminus (amino acid sequence CGGDYKDDDDK (SEQ ID NO:35)) was chemically coupled to purified HBcAg-Lys particles to provide an example of chemical crosslinking between a lysine residue and a cysteine residue. 600 μl of a 95% pure solution of HBcAg-Lys particles (2 mg/ml) were incubated for 30 minutes at room temperature with the heterobifunctional cross-linker N-Succinimidyl 3-(2-pyridyldithio) propionate (SPDP) (0.5 mM). After completion of the reaction, the mixture was dialyzed overnight against 1 liter of 50 mM Phosphate buffer (pH 7.2) with 150 mM NaCl to remove free SPDP. Then 500 μl of derivatized HBcAg-Lys capsid (2 mg/ml) were mixed with 0.1 mM FLAG peptide (containing an amino-terminal cysteine) in the presence of 10 mM EDTA to prevent metal-catalyzed sulfhydryl oxidation. The reaction was monitored through an increase in the optical density of the solution at 343 nm due to the release of pyridine-2-thione from SPDP upon reaction with the free cysteine of the peptide. The reaction of derivatized Lysine residues with the peptide was complete after approximately 30 minutes. The coupling efficiency was greater than 50%.

### Production and Coupling of Pili

Type-1 pili were produced from Escherichia coli as follows. E. coli strain W3110 was spread on LB (10 g/L tryptone, 5 g/L yeast extract, 5 g/L NaCl, pH 7.5, 1 % agar (w/v)) plates and incubated at 37° C. overnight. A single colony was then used to inoculate 5 ml of LB starter culture (10 g/L tryptone, 5 g/L yeast extract, 5 g/L NaCl, pH 7.5). After incubation for 24 hours under conditions that favor bacteria that produce Type-1 pili (37° C., without agitation), 5 shaker flasks containing 1 liter LB were inoculated with one milliliter of the starter culture. The bacterial cultures were then incubated for an additional 48 to 72 hours at 37° C. without agitation. Bacteria were then harvested by centrifugation (5000 rpm, 4° C., 10 minutes) and the resulting pellet was resuspended in 250 ml of 10 mM Tris/HCl, pH 7.5. Pili were detached from the bacteria by 5 minutes agitation in a conventional mixer at 17,000 rpm. After centrifugation for 10 minutes at 10,000 rpm at 4° C. the pili containing supernatant was collected, and 1 M MgCl₂ was added to a final concentration of 100 mM. The solution was kept at 4° C. for 1 hour, and the precipitated pili were then pelleted by centrifugation (10,000 rpm, 20 minutes, 4° C.). The pellet was then resuspended in 10 mM HEPES, pH 7.5, and the pilus solution was then clarified by a final centrifugation step to remove residual cell debris.

Coupling of FLAG to purified Type-1 pili of E. coli was accomplished using m-maleimidonbenzoyl-N-hydroxysulfosuccinimide ester (sulfo-MBS). 600 μl of a 95% pure solution of bacterial Type-1 pili (2 mg/ml) were incubated for 30 minutes at room temperature with the heterobifunctional cross-linker sulfo-MBS (0.5 mM). Thereafter, the mixture was dialyzed overnight against 1 liter of 50 mM Phosphate buffer (pH 7.2) with 150 mM NaCl to remove free sulfo-MBS. Then 500 μl of the derivatized pili (2 mg/ml) were mixed with 0.5 mM FLAG peptide (containing an amino-terminal Cysteine) in the presence of 10 mM EDTA to prevent metal-catalyzed sulfhydryloxidation. The non-coupled peptide was removed by size-exclusion-chromatography. The coupling efficiency was greater than 10%.

What is claimed is:

1. A composition comprising
(i) a carrier comprising a first attachment site;
(ii) a polypeptide selected from the group consisting of:
(a) at least one CH1 domain of an IgE molecule;
(b) at least one CH4 domain of an IgE molecule; and
(c) a combination of (a) and (b);
wherein said polypeptide contains or is bound to a second attachment site; and
wherein the first and second attachment sites are bound to each other.

2. The composition of claim 1, wherein the polypeptide lacks a IgE CH3 domain.

3. The composition of claim 1, wherein the carrier is selected from the group consisting of
(i) a virus,
(ii) a virus-like particle,
(iii) a bacteriophage,
(iv) a bacterial pilus,
(v) a viral capsid particle, and
(vi) a recombinant protein of (i), (ii), (iii), (iv) or (v).

4. The composition of claim 3, wherein the carrier is a virus-like particle derived from a virus selected from the group consisting of a Papilloma virus, a Rotavirus, a Norwalk virus, an Alphavirus, a Foot and Mouth Disease virus, a Retrovirus, a bacteriophage, and a Hepatitis B virus.

5. The composition of claim 1, wherein said first and second attachment sites comprise:
a) an antigen and an antibody or antibody fragment that specifically binds thereto,
b) biotin and avidin,
c) streptavidin and biotin,
d) a receptor and a ligand that binds to the receptor,
e) a ligand-binding protein and a ligand
f) interacting leucine zipper polypeptides,
g) an amino group and a chemical group reactive therewith,
h) a carboxyl group and a chemical group reactive therewith, or
i) a sulfhydryl group and a chemical group reactive therewith.

6. The composition of claim 1, wherein said first attachment site is bound to said second attachment site via a chemically-reactive amino acid.

7. The composition of claim 1, wherein the carrier is a polypeptide.

8. The composition of claim 1, wherein said first attachment site is bound to said second attachment site via a peptide bond, thereby providing a fusion protein comprising the polypeptide and the carrier.

9. The composition of claim 1, wherein said first attachment site comprises all or a portion of protein A.

10. The composition of claim 1, wherein said second attachment site comprises all or a portion of an immunoglobulin (Ig) variable region.

11. The composition of claim 1, wherein the polypeptide comprises at least two CH4 domains.

12. The composition of claim 1, wherein the polypeptide comprises at least two CH1 domains.

13. The composition of claim 1, wherein the polypeptide comprises at least two domains selected from the group consisting of a CH 1 domain and a CH4 domain, and the polypeptide further comprises one or more linkers covalently linking the domains.

14. The composition of claim 1, wherein said first attachment site comprises all or a portion of protein L.

15. The composition of claim 1, wherein the carrier comprises one or more epitopes of a T helper cell.

16. The composition of claim 1, wherein the IgE molecule is a human IgE molecule.

17. The composition of claim 1, wherein said second attachment site comprises all or a portion of a rodent IgG CH2 domain and all or a portion of a rodent IgG CH3 domain.

18. The composition of claim 1, wherein the carrier is a non-human protein.

19. The composition of claim 10, wherein the Ig variable region is a non-human Ig variable region.

20. The composition of claim 1 further comprising an adjuvant.

21. A polynucleotide encoding the fusion protein of claim 8.

22. A gene comprising the polynucleotide of claim 21.

23. A vector comprising the gene of claim 22.

24. A cell comprising the vector of claim 23.

25. A method for producing the fusion protein of claim 8, comprising inserting a vector containing a polynucleotide sequence encoding the fusion protein into a cell, and maintaining the cell under conditions such that the fusion protein is expressed.

26. A method for eliciting an immune response in a mammal, the method comprising administering to the mammal an immunogenic amount of the composition of claim 1.

27. A method for eliciting an immune response in a mammal, the method comprising administering to the mammal an immunogenic amount of the polynucleotide of claim 21.

28. A method for treating or inhibiting an IgE-mediated disorder in a mammal, the method comprising administering to a mammal in need thereof an effective amount of the composition of claim 1.

29. A method for treating or inhibiting an IgE-mediated disorder in a mammal, the method comprising administering to a mammal in need thereof an effective amount of the polynucleotide of claim 21.

30. The method of claim 28, wherein the IgE-mediated disorder comprises anaphylactic shock.

31. The method of claim 28, wherein the IgE-mediated disorder comprises allergic rhinitis or conjunctivitis.

32. The method of claim 31, wherein the IgE-mediated disorder comprises an allergic reaction to an allergen selected from the group consisting of fur, dust, and food.

33. The method of claim 31, wherein the IgE-mediated disorder comprises an asthmatic reaction.

34. The method of claim 31, wherein the IgE-mediated disorder comprises eczema or urticaria.

35. The composition of claim 1, wherein said first attachment site is bound to said second attachment site via a heterobifunctional cross-linking agent.

36. The composition of claim 35, wherein said agent comprises a N-hydroxy-succinimide ester group and a maleimide group.

37. The composition of claim 36, wherein said agent is ε-maleimidocaproic acid N-hydroxy-succinimide ester.

38. The composition of claim 36, wherein said N-hydroxy-succinimide ester group is chemically coupled to an amino moiety of a lysine group on said second attachment site; and
wherein said maleimide group is chemically coupled to the thiol moiety of a cysteine group on said first attachment site.

39. The composition of claim 36, wherein said N-hydroxy-succinimide ester group is chemically coupled to an amino moiety of a lysine group on said first attachment site; and
wherein said maleimide group is chemically coupled to the thiol moiety of a cysteine group on said second attachment site.

40. A cell comprising at least one isolated polypeptide selected from the group consisting of:
(a) one or a plurality of CH1 domains of an IgE molecule;
(b) one or a plurality of CH4 domains of an IgE molecule; and
(c) a combination of one or a plurality of CH1 domains of an IgE molecule and one or a plurality of CH4 domains of an IgE molecule.

41. The cell of claim 40, wherein said polypeptide consists of one or a plurality of CH1 domains of an IgE molecule, wherein each of said one or a plurality of CH1 domains is an amino acid sequence at least 95% identical to a sequence selected from the group consisting of:
(a) amino acids 1-110 of SEQ ID NO:1;
(b) amino acids 1-105 of SEQ ID NO:1;
(c) amino acids 5-105 of SEQ ID NO:1; and
(d) amino acids 5-95 of SEQ ID NO:1.

42. The cell of claim 40, wherein said polypeptide consists of one or a plurality of CH4 domains of an IgE molecule, wherein each of said one or a plurality of CH4 domains is an amino acid sequence at least 95% identical to a sequence selected from the group consisting of:
(a) amino acids 313-428 of SEQ ID NO:1;
(b) amino acids 313-425 of SEQ ID NO:1;
(c) amino acids 317-428 of SEQ ID NO:1; and
(d) amino acids 317-425 of SEQ ID NO:1.

43. The cell of claim 40, wherein said polypeptide consists of said combination, wherein said combination consists of
(i) one or a plurality of CH1 domains of an IgE molecule, wherein each of said one or a plurality of CH1 domains is an amino acid sequence at least 95% identical to a sequence selected from the group consisting of:
(a) amino acids 1-110 of SEQ ID NO:1;
(b) amino acids 1-105 of SEQ ID NO:1;
(c) amino acids 5-105 of SEQ ID NO:1; and
(d) amino acids 5-95 of SEQ ID NO:1;
and
(ii) one or a plurality of CH4 domains of an IgE molecule, wherein each of said one or a plurality of CH4 domains is an amino acid sequence at least 95% identical to a sequence selected from the group consisting of:
(a) amino acids 313-428 of SEQ ID NO:1;
(b) amino acids 313-425 of SEQ ID NO:1;
(c) amino acids 317-428 of SEQ ID NO:1; and
(d) amino acids 317-425 of SEQ ID NO:1.

44. The composition of claim 5, wherein said first attachment site is bound to said second attachment site via a cross-linking agent.

45. The composition of claim 44, wherein said crosslinking agent is a heterobifunctional cross-linking agent.

46. The composition of claim 45, wherein an amino group is covalently bound to a heterobifunctional cross-linking agent covalently bound to a sulfhydryl group.
